(19) United States
(12) Patent Application Publication
(10) Pub. No.: US 2016/0032359 A1
(43) Pub. Date: Feb. 4, 2016

(54) Methods for Generating Nucleic Acid Molecule Fragments Having a Customized Size Distribution

(71) Applicant: DANA-FARBER CANCER INSTITUTE, INC., Boston, MA (US)

(72) Inventors: Keith L. Ligon, Brookline, MA (US); Azra H. Ligon, Brookline, MA (US); Justin Craig, Cambridge, MA (US)

(73) Assignee: Dana-Farber Cancer Institute, Inc., Boston, MA (US)

(21) Appl. No.: 14/776,126

(22) PCT Filed: Mar. 12, 2014
(86) PCT No.: PCT/US14/24598

§ 371 Date: Sep. 14, 2015

Related U.S. Application Data
(60) Provisional application No. 61/788,006, filed on Mar. 15, 2013.

(51) Int. Cl. C12Q 1/68

(52) U.S. Cl. CPC C12Q 1/6806

(57) ABSTRACT

The invention provides methods for generating nucleic acid molecule fragments having a customized distribution. In one aspect, a method of generating nucleic acid fragments having a customized fragment size distribution is provided comprising obtaining a master pool of nucleic acid molecules to be fragmented; fragmenting at least two independent aliquots of the master pool of nucleic acid molecules in separate reactions, wherein the fragmentation conditions are identical except for a single variable.

### CROSS-REFERENCE TO RELATED APPLICATIONS

This application claims the benefit of U.S. Provisional Application No. 61/788,006, filed on Mar. 15, 2013; the entire content of said application is incorporated herein in its entirety by this reference.

### BACKGROUND OF THE INVENTION

Tumor-specific genomic aberrations are of great diagnostic and prognostic value. In addition, these aberrations are increasingly useful in selecting targeted therapies for individual patients (Corless (2011) Science 334:1217-1218). Current assays to establish copy number changes in clinical oncology are based on fluorescence in situ hybridization (FISH) and polymerase chain reaction (PCR) strategies designed to detect individual genomic alterations. However, large-scale cancer genome analyses continue to uncover specific aberrations in multiple cancers, and this, in turn, has driven the need for multiplex copy number testing in cancer research and clinical practice (Beroukhim et al. (2010) Nature 463:899-905; Cancer Genome Atlas Research Network (2011) Nature 474:609-615; and Cancer Genome Atlas Research Network (2008) Nature 455:1061-1068). Genome-wide technologies to determine copy number changes, such as array comparative genomic hybridization (aCGH) and single nucleotide polymorphism (SNP) arrays, were among the first whole-genome technologies developed (Pinkel et al. (1998) Nat. Genet. 20:207-211). More recently, these technologies have been able to query the genome at intra-exon resolution and, as demonstrated in recent large-scale projects such as the Cancer Genome Atlas (Cancer Genome Atlas Research Network (2008) Nature 455:1061-1068), can offer not only high-throughput analysis but also robust genome-wide copy number data.

Copy number analysis assays have been widely used in the research setting. Most of these basic research studies use frozen tumor samples that yield high-quality, intact DNA. The application of similar assays in clinical trials and in the routine clinical diagnosis of tumors has been unexpectedly slow, however. The greatest impediment to clinical implementation has been the technical challenges encountered during the processing and analysis of formalin-fixed paraffin-embedded (FFPE) samples, the mainstay of pathology department workflow. The inconsistent aCGH data that often results from FFPE samples is generally attributed to reduced DNA integrity. The relatively poor quality and variable results obtained from FFPE aCGH are particularly concerning because aCGH requires significantly more tissue than FISH or colorimetric in situ hybridization (CISH), both of which are performed routinely using FFPE specimens.

Early attempts at aCGH analysis of FFPE specimens were hindered because of inadequate sensitivity and specificity (McSherry et al. (2007) Clin. Genet. 72:441-447 and Pinkel and Albertson (2005) Nat. Genet. 37:S11-S17). Improvements in DNA extraction protocols (Paris et al. (2007) The Prostate 67:1447-1455; van Beers et al. (2006) Brit. J. Canc. 94:333-337; Wessels et al. (2002) Canc. Res. 62:7110-7117; and Alers et al. (1997) Lab. Invest. 77:437-448), labeling techniques (van Gijlswijk et al. (2001) Exp. Rev. Mol. Diagnost. 1:81-91), and aCGH platforms (Pinkel et al. (1998) Nat. Genet. 20:207-211; Brennan et al. (2004) Canc. Res. 64:4744-4748; and Barrett et al. (2004) Proc. Natl. Acad. Sci. USA 101:17765-17770) subsequently facilitated the analysis of FFPE samples in the research setting. To date, several studies have suggested that informative aCGH data can be generated from FFPE tissues (Paris et al. (2007) The Prostate 67:1447-1455; van Beers et al. (2006) Brit. J. Canc. 94:333-337; Devries et al. (2005) J. Mol. Diag. 7:65-71; Johnson et al. (2006) Lab. Invest. 86:968-978; Maher et al. (2006) Canc. Res. 66:11502-11513; Paris et al. (2003) Amer. J. Pathol. 162:763-770; Hostetter et al. (2010) Nucl. Acids Res. 38:e9; Mohapatra et al. (2011) Acta Neuropathol. 121:529-543; and Harada et al. (2011) J. Mol. Diagnost. 13:541-548), although reports in the literature indicate that one-third of FFPE specimens generate suboptimal aCGH results using standard methods (van Beers et al. (2006) Brit. J. Canc. 94:333-337). This is particularly relevant for older specimens such as those used in retrospective analysis (e.g., clinical trials cohorts) (Pinkel and Albertson (2005) Nat. Genet. 37:S11-S17; Devries et al. (2005) J. Mol. Diag. 7:65-71; Johnson et al. (2006) Lab. Invest. 86:968-978; Hostetter et al. (2010) Nucl. Acids Res. 38:e9; and Braggio et al. (2011) Clin. Canc. Res. 17:4245-4253).

Although the compromised integrity of DNA extracted from FFPE tissues has long been suspected as the source of the technical difficulties with FFPE aCGH, direct demonstration of this causal relationship and how to remedy it has proven challenging (Pinkel and Albertson (2005) Nat. Genet. 37:S11-S17). Several quality control (QC) metrics have been proposed for prospectively determining DNA suitability for aCGH. For each of these methods DNA degradation has generally been assessed using measurements of DNA size. Examples include: (1) multiplex-PCR to exclude DNA samples that fail to produce minimum size lengths; (2) gel electrophoresis to exclude DNA samples with average fragment size below a given minimum molecular weight; and (3) whole genome amplification (WGA) to exclude DNA samples that result in low DNA yields (van Beers et al. (2006) Brit. J. Canc. 94:333-337; Johnson et al. (2006) Lab. Invest. 86:968-978; Harada et al. (2011) J. Mol. Diagnost. 13:541-548; Buffart et al. (2007) Cell. Oncol. 29:351-359; and Alers et al. (1999) Genes, Chrom. Canc. 25:301-305). These studies assess DNA integrity prior to DNA labeling and subsequent hybridization. The specific conditions involved in DNA labeling—whether enzymatic- or chemical-based—cause additional fragmentation and physical modification of DNA (Alers et al. (1999) Gene. Chrom. Canc. 25:301-305 and Gustafson et al. (1993) Gene 123:241-244). Therefore, any quality assessments performed prior to these steps do not evaluate the integrity of the DNA that is actually being hybridized to the array. Furthermore, these metrics help prevent assay failure without offering methods for improving the performance of samples known to contain suboptimal DNA. If aCGH or other assays that would benefit from processing nucleic acid inputs having a defined and/or uniform size of useful specimens, such as FFPE specimens, is to become feasible clinically, the process must be standardized to eliminate sample-to-sample variability as well as to significantly enhance both data quality and reproducibility (Idbaih et al. (2010) Brain Pathol. 20:28-38 and Nowak et al. (2007) Genet. Med. 9:585-595).

Moreover, many useful assays would benefit from processing nucleic acid inputs having a defined and/or uniform size, such as hybridization-based nucleic acid assays (e.g., single nucleotide polymorphisms (SNP) and nanostring assays), nucleic acid sequencing assays (e.g., next-generation sequencing and whole exome assays), and the like. Given the fact that such assays are expensive and healthcare and diagnostic service providers are increasingly under pressure to reduce or capitate costs, a great need exists for methods to generate nucleic acids having a defined and/or uniform size, especially from samples that would otherwise be discarded as having unacceptable quality according to known nucleic acid manipulation techniques.

### SUMMARY OF THE INVENTION

The present invention overcomes the long-felt difficulties in generating nucleic acid molecule fragments having a customized size distribution tailored to a given sample.

In one aspect, a method of generating nucleic acid fragments having a customized fragment size distribution is provided comprising: a) obtaining a master pool of nucleic acid molecules to be fragmented; b) fragmenting at least two independent aliquots of the master pool of nucleic acid molecules in separate reactions, wherein the fragmentation conditions of each separate reaction are identical except for a single variable; c) determining the nucleic acid molecule fragment size distribution from each aliquot; d) plotting each nucleic acid molecule fragment size distribution result on a graph as a function of a value of the single variable for each aliquot; e) fitting a curve to the plotted nucleic acid molecule fragment size distribution results; f) identifying the value of the single variable necessary to obtain the desired nucleic acid molecule fragment size distribution on the curve; and g) fragmenting the master pool of nucleic acid molecules or an aliquot thereof, wherein the fragmentation conditions are performed using the identified value of the single variable necessary to obtain the desired nucleic acid molecule fragment size distribution, to thereby generate nucleic acid fragments having a customized fragment size distribution.

In any embodiment of the method, the method can be adapted or modified according to variations described herein or any combination of such variations thereof. In one embodiment, step b) further comprises treating the nucleic acid molecules or fragments thereof with at least one additional nucleic acid modifying reaction to modify or simulate the modification of the nucleic acid molecules or fragments thereof (e.g., a nucleic acid labeling reaction). In another embodiment, the at least one additional nucleic acid modifying reaction or simulated reaction thereof is performed before, simultaneously with, or after the fragmentation reaction. In still another embodiment, step g) further comprises treating the nucleic acid fragments with the at least one additional nucleic acid modifying reaction of step b) (e.g., a nucleic acid labeling reaction). In yet another embodiment, the at least one additional nucleic acid modifying reaction is performed before, simultaneously with, or after the fragmentation reaction. In another embodiment, the nucleic acid fragments having a customized fragment size distribution are used in a nucleic acid hybridization, sequencing, or amplification assay and step b) further comprises treating the nucleic acid molecules or fragments thereof with every nucleic acid processing step required for the assay prior to hybridization, sequencing, or amplification, or modeling each step thereof. In still another embodiment, the nucleic acid processing or modeled processing steps are performed before, simultaneously with, or after the fragmentation reaction. In yet another embodiment, step g) further comprises treating the nucleic acid fragments thereof with every nucleic acid processing step required for the assay prior to hybridization, sequencing, or amplification. In another embodiment, the nucleic acid processing steps are performed before, simultaneously with, or after the fragmentation reaction. In still another embodiment, the nucleic acid molecules are obtained from a sample selected from the group consisting of formalin-fixed paraffin-embedded (FFPE), paraffin, frozen, and fresh samples. In yet another embodiment, the sample contains a tissue specimen and the tissue specimen was present in the sample for more than one year after isolation from a host organism. In another embodiment, the nucleic acid molecules to be fragmented are selected from the group consisting of genomic DNA, cDNA, double-stranded DNA, single-stranded DNA, double-stranded RNA, single-stranded RNA, and messenger RNAs. In still another embodiment, the nucleic acid molecules to be fragmented are fragmented by heat fragmentation, enzymatic digestion, shearing, mechanical crushing, chemical treatment, nebulizing, or sonication. In yet another embodiment, the single variable is selected from the group consisting of time, temperature, pressure, shear force, reagent amount, reagent concentration, reagent activity, acoustic wavelength, and acoustic frequency. In another embodiment, the at least two aliquots of step b) are performed simultaneously or sequentially. In still another embodiment, step b) is performed with at least 3 or at least 4 aliquots. In yet another embodiment, the fragment size distribution is measured as the mode, mean, or median of fragment lengths. In another embodiment, the curve is fit using a linear model, an exponential decay model, or an inverse power law. In still another embodiment, the inverse power law is given by the mathematical formula,

$$f ( t ) = {θ}_{1} + \frac{{θ}_{2}}{{( t + {θ}_{3} )}^{{θ}_{4}}} ,$$

where f(t) is the mode DNA fragment size, t is the single variable for each aliquot representing time of heat fragmentation, and θ₁, θ₂, θ₃, and θ₄ are constant parameters unique for each aliquot. In yet another embodiment, the constant parameters, θ₁, θ₂, θ₃, and θ₄, are determined using iterative least squares non-linear regression. In another embodiment, a method of generating nucleic acid fragments having customized and essentially identical fragment size distributions from each of at least two independent master pools of nucleic acid molecules to be fragmented is provided comprising performing the a method of the present invention, or adaptations, modifications or any combinations thereof as described herein, using at least two master pools of nucleic acid molecules.

### BRIEF DESCRIPTION OF FIGURES

FIGS. 1A-1H shows that “matching” DNA fragment size distributions are necessary for optimal aCGH data. FIGS. 1A, 1C, 1E, and 1G show agarose gel electrophoresis images and ImageJ gel intensity analysis plots of reference gDNA (Promega) after heat fragmentation. Mode fragment size is indicated with arrowed lines (base pairs; bp) relative to DNA ladder. Heat times were adjusted to produce four mode fragment size combinations (225/225, 525/225, 525/140, 225/140). FIGS. 1B, 1D, 1F, and 1H show a plot of results from chromosome 1 following self-hybridization of specific combinations of mode size. Differentially labeled aliquots (cy5/cy3) were coded according to log₂ ratio range: log₂ ratio<−0.3; −0.3≦log₂ ratio≦3; and log₂ ratio>0.3. Data quality was assessed by dLRsd on Agilent 180 K arrays.

FIG. 2A-2I shows a determination of optimal size among matched DNA fragment size distributions. FIGS. 2A, 2C, 2E, and 2G show agarose gel electrophoresis of reference gDNA (Promega) aliquots after various heat fragmentation times shown adjacent to ImageJ gel analysis of same lanes. Molecular weight is indicated in bp. The mode fragment size of each smear, as measured with ImageJ, is indicated with arrowed lines. FIGS. 2B, 2D, 2F, and 2H show Agilent 180 K array results of self-hybridizations using reference gDNA (left) and characterized by matching fragment size distributions (FIG. 2B; 250/250, FIG. 2D; 315/315, FIG. 2F; 400/400, and FIG. 2H; 525/525). Log₂ ratios for signal intensities of differentially labeled aliquots (cy5/cy3) are plotted for probes corresponding to chromosome 1 according to log₂ ratio (log₂ ratio<−0.3; −0.3≦log₂ ratio≦0.3; and log₂ ratio>0.3). Data quality was assessed by dLRsd. FIG. 2I shows the mean dLRsd of duplicate (n=5) or triplicate (n=2) size-matched self-hybridizations representing seven fragment size distributions plotted by mode fragment length (225, 250, 315, 400, 525, 625, and 680 bp). Error bars are indicated as the standard error of the mean (SEM).

FIGS. 3A-3F show that DNA fragmentation and thermodegradation are unpredictably variable. FIG. 3A shows a gel electrophoresis image of DNA extracted from 22 FFPE tissue specimens stored in paraffin from one to 13 years. FIG. 3B shows mode fragment sizes of samples in FIG. 3A plotted by age of paraffin block. Linear regression of the data is indicated by the dashed line. FIG. 3C shows a gel electrophoresis image of DNA from six FFPE specimens intact prior to labeling (i), after ULS labeling only (0), or after ULS labeling plus 1 min heat fragmentation (1). FIG. 3D shows the mode fragment size of lanes marked 0 and 1 plotted for the six FFPE samples from the gel shown in FIG. 3C. FIG. 3E shows a gel electrophoresis image of DNA from three frozen specimens with i, 0, and 1 indicating the same conditions as in FIG. 3C, and samples after ULS labeling conditions plus 2 min heat fragmentation (2). FIG. 3F shows a plot of mode fragment size for lanes marked 0, 1, and 2 plotted for the three frozen samples shown in FIG. 3E.

FIGS. 4A-4F show that a fragmentation simulation method (FSM) enables accurate prediction and precise control of labeled DNA fragment sizes. FIGS. 4A and 4D show gel images of DNA from three FFPE specimens (FIG. 4A) or three frozen specimens (FIG. 4D) either intact, (i), after ULS labeling conditions only, (0), or ULS labeling conditions and 0.5, 1, 2, 4, 6, or eight minutes heat fragmentation (0.5, 1, 2, 4, 6, 8). FIGS. 4B and 4E show FSM regression curves fit to data from each sample from FIGS. 4A and 4D by utilizing the mode fragment size of lanes in FIG. 4A or FIG. 4D, respectively, as data points. The intersection with target size (dashed line) reveals FSM prediction for optimal time of heat fragmentation for each sample. FIGS. 4C and 4F show agarose gel electrophoresis results of samples in FIG. 4A or FIG. 4D after heat fragmentation for time predicted by FSM in FIG. 4B or FIG. 4E and ULS labeling conditions, shown adjacent to ImageJ gel analysis of same lanes. The mode fragment size of each smear, as measured with ImageJ, is indicated by arrows and solid horizontal lines. For FIGS. 4A-4F, the vertical axes indicate DNA bp.

FIGS. 5A-5D show that application of a FSM ULS method to FFPE samples creates equivalent results to those from fresh-frozen samples. FIG. 5A is a plot showing dLRsd for 122 FFPE tumor specimens processed according to either standard ULS or FSM ULS protocols and analyzed on Agilent 1 M arrays. FIG. 5B shows data quality (dLRsd) from Figure A plotted by FFPE block age and method. Dashed lines indicate linear regression. The statistics indicate the magnitude and significance of correlation between block age and aCGH data quality. FIG. 5C shows the quality (dLRsd) of Agilent 1 M aCGH data of 78 fresh-frozen tissue specimens or frozen tumorsphere cell cultures processed according to either standard ULS or FSM ULS protocols. FIG. 5D shows FFPE and frozen FSM ULS subsets from FIGS. 5A and 5C compared to 206 fresh-frozen GBM specimens analyzed on Agilent 244 k arrays from the glioblastoma TCGA study. Statistical significance was assessed by t test and ANOVA. (****; p<0.0001, ns; p>0.05), and error bars indicate the mean and standard deviation. Additional QC metrics data for all samples are provided in Table 2.

FIGS. 6A-6H show that size matching using FSM is a more critical determinant of array quality than other known variables. For each of the figures, the probe log₂ ratio (signal intensity test DNA/signal intensity reference DNA) data is plotted for a single chromosome (chr.13 or chr.1) from eight Agilent 1 M arrays and are presented in log₂ ratio ranges (log₂ ratio<−0.3; −0.3<log₂ ratio≦0.3; and log₂ ratio>0.3). FIGS. 6A-6C show chromosome 13 plotted log₂ ratios from representative profiles of three Agilent 1 M arrays of a single FFPE GBM specimen (GBM1) processed with the FSM ULS protocol (FIG. 6A), standard ULS protocol (FIG. 6B), or FSM ULS protocol after altered proteinase K digestion during DNA extraction (FIG. 6C). The plotted log: ratio data for all chromosomes is provided in FIG. 8. FIGS. 6D-6H show chromosome 1 plotted log ratios from representative profiles of five Agilent 1 M arrays of a single FFPE GBM specimen (GBM2) processed using the FSM ULS protocol, with reduced DNA input in FIG. 6E and FIG. 6F. FIG. 9 and FIG. 10 provide detailed copy number analysis. FIG. 6G shows that increased hybridization time improved quality to a modest degree. FIG. 6H shows that the use of FFPE brain tissue as reference DNA did not significantly improve results (dLRsd of 0.21 vs. 0.20 for standard reference).

FIGS. 7A-7C show that FSM ULS probe level data demonstrates greater sensitivity and specificity than standard ULS probe level data. Female FFPE tumor DNA from sample GBM1 was hybridized with normal male reference DNA (Promega) on Agilent 1 M arrays using either the FSM ULS or Standard ULS protocols. Log₂ ratio data from X chromosome (XX/XY) and chromosome 8 (copy neutral) were compared for each array. FIG. 7A shows receiver operating characteristic (ROC) curves plotting sensitivity and specificity across a range of log 2 ratio thresholds and indicating that aberrant (X chromosome) probe values are more readily distinguished from non-aberrant (chromosome 8) probe values in FSM ULS data than in Standard ULS data. AUC indicates the area under the respective ROC curve. FIGS. 7B and 7C show that, given optimized log₂ ratio thresholds defined by ROC analysis (dashed vertical line), log₂ ratio frequency distributions were plotted as a curve and false positive rate (FPR) and false negative rate (FNR) were calculated. FPR is defined as proportion of copy neutral (chr8) probe values incorrectly classified as aberrant and FNR is defined as proportion of aberrant (Xchr) probe values incorrectly classified as copy neutral.

FIG. 8 shows a whole genome view of Agilent 1 M array data for FFPE sample GBM1 prepared by FSM versus standard ULS methods. Log₂ ratios were plotted for three Agilent 1 M arrays hybridized using either the FSM ULS protocol (left column of each chromosome), the standard ULS protocol (middle column of each chromosome), or the FSM ULS protocol and DNA extracted with reduced duration Proteinase K digestion (right column of each chromosome) as in FIGS. 6A-6C and are presented in log₂ ratio ranges (log₂ ratio<−0.3; −0.3≦log₂ ratio≦0.3; and log₂ ratio>0.3). FSM methods yield lower noise across the whole genome compared to standard ULS even with shorter Proteinase K digestion.

FIGS. 9A-9C show that a FSM ULS protocol enables robust aberration detection with as little as 10% of recommended FFPE DNA input. FFPE sample GBM2, as shown in FIGS. 6D-6H, were hybridized to Agilent 1 M arrays using 100% (2.0 μg), 75% (1.5 μg), 50% (1.0 μg), 25% (0.5 μg), and 10% (0.2 μg) of the recommended DNA input. Aberration analysis utilized the Agilent Genomic Workbench 6.5 algorithm ADM-2 (threshold=7.0, probes≧7, minimum average absolute log₂ ratio ≧0.35). FIG. 9A shows a whole genome representation of aberrations detected in Agilent 1 M aCGH data produced from varying DNA inputs. DNA input lines farthest from the X-axis, both above and below the X-axis, correspond to 2.0 ug DNA and descend in order as layered bands stretching across the graph with decreasing distance from the X-axis according to 1.5 μg DNA, 1.0 μg DNA, 0.5 μg DNA, and 0.2 μg DNA, respectively and in that order, towards the X-axis. FIG. 9B shows that a summary of detected aberrations revealed a ˜96% (26/27) concordance between aberrations detected using 10% of standard DNA input and 100% of standard DNA input, though disparities in interval breakpoints increase significantly with lower amounts of input DNA. FIG. 9C shows chromosome 1 log₂ ratios plotted for five Agilent 1 M arrays of FFPE GBM specimen GBM2 processed using the FSM ULS protocol and decreasing DNA inputs and are presented in log₂ ratio ranges (log₂ ratio<−0.3; −0.3≦log₂ ratio≦0.3; and log₂ ratio>0.3). While higher dLRsd indicates poorer quality in the 25% and 10% input arrays, similar aberrations (see the bold, two-part broken lines above and below the X-axis) detected in the higher DNA input arrays suggest the utility of limited DNA inputs when detection of very focal (<100 kb) copy number alterations and precise breakpoints is not necessary.

FIGS. 10A-10D show the effect of a FSM ULS protocol and DNA input on Agilent 1 M aCGH probe level sensitivity and specificity. The data generated from FFPE sample GBM2 shown in FIGS. 6D-6H and Agilent 1 M arrays using 100% (2.0 μg), 75% (1.5 μg), 50% (1.0 μg), 25% (0.5 μg), and 10% (0.2 μg) of the recommended FFPE DNA input were used for the analysis. The Agilent Genomic Workbench 6.5 algorithm ADM-2 (threshold=7.0, probes ≧7, minimum average absolute log₂ ratio ≧0.35) was utilized to define regions of single copy gain (0.35≦average log₂ ratio ≦0.58), single copy loss (−1.0≦average log₂ ratio ≦−0.35), and non-aberrant copy neutral regions in GBM2 FSM extended hybridization data (FIG. 6G), which were then used to standardize receiver operating characteristic (ROC) analysis. FIGS. 10A and 10C show ROC curves plotting sensitivity and I-specificity across a range of log₂ ratio thresholds and demonstrate that probe values in regions of either single copy gain (FIG. 10A) or single copy loss (FIG. 10C) are more readily distinguished from probe values in copy neutral regions with greater DNA input (AUC indicates area under respective ROC curve). FIGS. 10B and 10D show that, given ROC optimized log₂ ratio thresholds (dashed vertical lines) for detecting single copy gain (FIG. 10B) or single copy loss (FIG. 10D) in data from each DNA input, log₂ ratio frequency distributions were plotted for probes in copy neutral regions and either regions of single copy gain (FIG. 10B) or single copy loss (FIG. 10D). False positive rates (FPR) and false negative rates (FNR) were calculated as follows: FPR is defined as proportion of probe values in copy neutral regions incorrectly classified as aberrant, FNR is defined as proportion of probe values in regions of gain or loss incorrectly classified as copy neutral. While the added information of genomic location and measurements from multiple probes enable algorithmic aberration detection with similar results across all DNA inputs (see FIG. 9), significantly higher probe level FPR and FNR were observed at lower DNA inputs and indicate compromised array level resolution.

FIG. 11 shows a representative schematic overview of the proposed methods and timeline for FSM ULS processing of FFPE specimens and use in, for example, aCGH assays. Following DNA extraction, the workflow and protocol for preparation of fresh or frozen samples is identical to FFPE workflow shown.

FIG. 12 shows a predicted hierarchy of known variables contributing to aCGH data quality.

### DETAILED DESCRIPTION OF THE INVENTION

In general, valuable samples used to extract nucleic acids for downstream analyses are homogeneously processed according to a standard assay protocol without regard to customized procedures for each sample. An important quality control (QC) measurement is nucleic acid size of nucleic acid samples since performance of many nucleic acid analysis technologies is dependent on the nucleic acid size of input nucleic acids. Since such downstream analyses typically use expensive reagents and incur significant costs to perform, samples not meeting nucleic acid size requirements and/or other QC measurements are simply discarded on the assumption that the sample preparation was intrinsically unsuitable for the desired application. By contrast, the present invention is based in part on the discovery that such standard nucleic acid assay protocols (e.g., nucleic acid fragmentation protocols) result in significantly variable results in any given sample and that a simulation model can be performed for each sample to customize the assay protocol for each sample in order to generate nucleic acid molecules having a customized size distribution. The present invention further provides, in part, methods for generating nucleic acid molecules having customized size distributions and which size distributions are uniform across multiple nucleic acid samples since it has been determined herein that such samples having paired or matched nucleic acid size distributions significantly improves the results of competitive hybridization-based nucleic acid analyses using such samples.

### A. Samples and Preparation of Nucleic Acid Molecules for Fragmentation

The methods described herein use nucleic acid molecules to generate fragments thereof having a customized fragment size distribution. The term “nucleic acid molecules” or “nucleic acids” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. The terms “ribonucleic acid” and “RNA” as used herein mean a polymer composed of ribonucleotides. The terms “deoxyribonucleic acid” and “DNA” as used herein mean a polymer composed of deoxyribonucleotides. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. In one embodiment, the nucleic acid molecules to be fragmented are derived from genomic DNA. Such genomic DNA can comprise exome DNA, i.e., a subset of whole genomic DNA enriched for transcribed sequences which contains the set of exons in a genome. In further embodiments, the target nucleic acids comprise a transcriptome (i.e., the set of all mRNA or “transcripts” produced in a cell or population of cells), a methylome (i.e., the population of methylated sites and the pattern of methylation in a genome), a phosphorylome, and the like.

Nucleic acid molecules to be fragmented can be derived from a sample of material comprising such molecules, such as from biological sources. The term “sample” is used herein in a broad sense and is intended to include a variety of sources and compositions that contain nucleic acids. The sample may be a biological sample, but the term also includes other, for example, artificial samples which comprise nucleic acids. Exemplary samples include, but are not limited to, whole blood: blood products such as plasma or serum; red blood cells: white blood cells; buffy coat; swabs, including but not limited to buccal swabs, throat swabs, vaginal swabs, urethral swabs, cervical swabs, throat swabs, rectal swabs, lesion swabs, abcess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; pulmonary lavage; lung aspirates; tissues, including but not limited to, liver, spleen, kidney, lung, intestine, brain, heart, muscle, pancreas, cell cultures, plant tissues or samples, as well as lysates, extracts, or materials and fractions obtained from the samples described above or any cells and microorganisms and viruses that may be present on or in a sample and the like. Materials obtained from clinical or forensic settings that contain nucleic acids are also within the intended meaning of the term “sample.” In one embodiment, nucleic acid sources from subjects having a particular condition, such as cancer, can be used. Non-limiting examples of such samples include frozen tissue samples, fresh tissue samples, paraffin-embedded samples, and samples that have been preserved, e.g. formalin-fixed and paraffin-embedded (FFPE samples) or other samples that were treated with cross-linking fixatives such as, for example, glutaraldehyde. The methods according to the present invention are particularly useful for generating nucleic acid molecules having a customized size distribution from samples containing degraded or compromised nucleic acids (e.g., DNA and RNA). For example, biopsy samples from tumors are routinely stored after surgical procedures by FFPE samples, which may compromise DNA and/or RNA integrity.

The sample can be a biological sample derived from a human, animal, plant, bacteria or a fungus. The sample can be selected from the group consisting of cells, tissue, bacteria, virus and body fluids such as for example blood, blood products such as buffy coat, plasma and serum, urine, liquor, sputum, stool, CSF and sperm, epithelial swabs, biopsies, bone marrow samples and tissue samples, preferably organ tissue samples such as lung, kidney or liver. Furthermore, the skilled artisan will appreciate that lysates, extracts, or processed materials or portions obtained from any of the above exemplary samples are also within the scope of the term “sample.”

As described above, the term “sample” also includes processed samples such as preserved, fixed and/or stabilized samples. As described herein, suitable samples useful for extracting nucleic acid molecules to be fragmented according to the methods of the present invention described herein can contain biological material retrieved from a host organism of 1 year, 2 years, 3 years, 4 years, 5 years, 6 years, 7 years, 8 years, 9 years, 10 years, 1 years, 12 years, 13 years, 14 years, 15 years, 16 years, 17 years, 18 years, 19 years, 20 years, or longer before the methods of the present invention are applied.

A “master pool” of nucleic acid molecules to be fragmented refers to an initial stock of nucleic acids molecules whose sizes are larger than those desired. From this master pool, one or more aliquots can be generated by separating away a portion of the master pool for analysis without affecting the remaining nucleic acid molecules remaining in the master pool.

For those embodiments where biological samples are used to obtain the master pool, such as whole cells, viruses or other tissue samples being analyzed, it will typically be necessary to extract the nucleic acids from the material in order to generate the master pool. Accordingly, following sample collection, nucleic acids may be liberated from the collected cells, viral coat, etc., into a crude extract, followed by additional treatments to prepare the sample for subsequent operations, e.g., denaturation of contaminating (DNA binding) proteins, purification, filtration, desalting, and the like.

Liberation of nucleic acids from the sample cells or viruses, and denaturation of DNA binding proteins may generally be performed using well-known chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within the extraction chamber, a separate accessible chamber or externally introduced.

Alternatively, physical methods may be used to extract the nucleic acids and denature DNA binding proteins. U.S. Pat. No. 5,304,487, incorporated herein by reference in its entirety, discusses the use of physical protrusions within microchannels or sharp edged particles within a chamber or channel to pierce cell membranes and extract their contents. Combinations of such structures with piezoelectric elements for agitation can provide suitable shear forces for lysis. Such elements are described in greater detail with respect to nucleic acid fragmentation, below. More traditional methods of cell extraction may also be used, e.g., employing a channel with restricted cross-sectional dimension which causes cell lysis when the sample is passed through the channel with sufficient flow pressure.

In some embodiments, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysislextraction, including, e.g., subjecting cells to ultrasonic agitation, or forcing cells through microgeometry apertures, thereby subjecting the cells to high shear stress resulting in rupture.

Following extraction, it will often be desirable to separate the nucleic acids from other elements of the crude extract, e.g., denatured proteins, cell membrane particles, salts, and the like. Removal of particulate matter is generally accomplished by filtration, flocculation or the like. A variety of filter types may be readily incorporated into the device. Further, where chemical denaturing methods are used, it may be desirable to desalt the sample prior to proceeding to the next step. Desalting of the sample, and isolation of the nucleic acid may generally be carried out in a single step. e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art, may also be readily incorporated into the devices of the present invention, and is commercially available from, e.g., Pharmacia and Sigma Chemical.

The isolation and/or gel filtration/desalting may be carried out in an additional chamber, or alternatively, the particular chromatographic media may be incorporated in a channel or fluid passage leading to a subsequent reaction chamber. Alternatively, the interior surfaces of one or more fluid passages or chambers may themselves be derivatized to provide functional groups appropriate for the desired purification, e.g., charged groups, affinity binding groups and the like, i.e., poly-T oligonucleotides for mRNA purification.

Alternatively, desalting methods may generally take advantage of the high electrophoretic mobility and negative charge of DNA compared to other elements. Electrophoretic methods may also be utilized in the purification of nucleic acids from other cell contaminants and debris. In one example, a separation channel or chamber of the device is fluidly connected to two separate “field” channels or chambers having electrodes, e.g., platinum electrodes, disposed therein. The two field channels are separated from the separation channel using an appropriate barrier or “capture membrane” which allows for passage of current without allowing passage of nucleic acids or other large molecules. The barrier generally serves two basic functions: first, the barrier acts to retain the nucleic acids which migrate toward the positive electrode within the separation chamber; and second, the barriers prevent the adverse effects associated with electrolysis at the electrode from entering into the reaction chamber (e.g., acting as a salt junction). Such barriers may include, e.g., dialysis membranes, dense gels, PEI filters, or other suitable materials. Upon application of an appropriate electric field, the nucleic acids present in the sample will migrate toward the positive electrode and become trapped on the capture membrane. Sample impurities remaining free of the membrane are then washed from the chamber by applying an appropriate fluid flow. Upon reversal of the voltage, the nucleic acids are released from the membrane in a substantially purer form. The field channels may be disposed on the same or opposite sides or ends of a separation chamber or channel, and may be used in conjunction with mixing elements described herein, to ensure maximal efficiency of operation. Further, coarse filters may also be overlaid on the barriers to avoid any fouling of the barriers by particulate matter, proteins or nucleic acids, thereby permitting repeated use.

In a similar aspect, the high electrophoretic mobility of nucleic acids with their negative charges, may be utilized to separate nucleic acids from contaminants by utilizing a short column of a gel or other appropriate matrix or gel which will slow or retard the flow of other contaminants while allowing the faster nucleic acids to pass.

In some embodiments, it may be desirable to extract certain species of nucleic acids, such as DNA or RNA, species based on size (e.g., genomic, plasmid, transcribed, small, micro, chromosomal, etc.), species based on strandedness (e.g., single stranded or double stranded), species based on composition (e.g., cDNA or cRNA), and the like. Conventional techniques for isolating desired nucleic acids can be used and are well known in the art for example as disclosed in Sambrook and Russell, Molecular Cloning: A Laboratory Manual and as described in the Examples.

Non-limiting, exemplary techniques include methods of using a cartridge supported with a nucleic acid-adsorbable membrane of silica, cellulose compound, or the like, precipitation with ethanol or precipitation with isopropanol, extraction with phenol-chloroform, and the like. Furthermore, there may be mentioned methods with solid-phase extraction cartridge, chromatography, and the like using ion-exchange resins, silica supports bonded with a hydrophobic substituent such as an octadecyl group, resins having a size-exclusion effect.

For example, it may be desirable to extract and separate messenger RNA from cells, cellular debris, and other contaminants. As such, the device of the present invention may, in some cases, include an mRNA purification chamber or channel. In general, such purification takes advantage of the poly-A tails on mRNA. In particular and as noted above, poly-T oligonucleotides may be immobilized within a chamber or channel of the device to serve as affinity ligands for mRNA. Poly-T oligonucleotides may be immobilized upon a solid support incorporated within the chamber or channel, or alternatively, may be immobilized upon the surface(s) of the chamber or channel itself. Immobilization of oligonucleotides on the surface of the chambers or channels may be carried out by methods described herein including, e.g., oxidation and silanation of the surface followed by standard DMT synthesis of the oligonucleotides. In operation, the lysed sample is introduced into this chamber or channel in an appropriate salt solution for hybridization, whereupon the mRNA will hybridize to the immobilized poly-T. After enough time has elapsed for hybridization, the chamber or channel is washed with clean salt solution. The mRNA bound to the immobilized poly-T oligonucleotides is then washed free in a low ionic strength buffer. The surface area upon which the poly-T oligonucleotides are immobilized may be increased through the use of etched structures within the chamber or channel, e.g., ridges, grooves or the like. Such structures also aid in the agitation of the contents of the chamber or channel, as described herein. Alternatively, the poly-T oligonucleotides may be immobilized upon porous surfaces, e.g., porous silicon, zeolites, silica xerogels, cellulose, sintered particles, or other solid supports.

### B. Nucleic Acid Fragmentation

Nucleic acid molecules to be fragmented to a customized (i.e. desired) size can be generated using conventional techniques including heat fragmentation (thermodegradation), enzymatic digestion, shearing, mechanical crushing, chemical treatment, nebulizing, sonication, and the like.

These fragmentation methods are generally random in that the generated fragments of a polynucleotide molecule is in a non-ordered fashion. Such fragmentation methods are known in the art and utilize standard methods (Sambrook and Russell, Molecular Cloning, A Laboratory Manual, third edition). By contrast, generating smaller fragments of a larger piece of nucleic acid by specifically amplifying smaller fragments, such as by PCR amplification, is not equivalent to fragmenting the larger piece of nucleic acid because the larger piece of nucleic acid sequence remains intact (i.e., is not fragmented by the PCR amplification). The random fragmentation is designed to produce fragments irrespective of the sequence identity or position of nucleotides comprising and/or surrounding the break. More particularly the random fragmentation is by physical means.

Thermodegradation involves heat-based fragmentation of nucleic acids. In one embodiment, temperatures of 8° C., 85° C., 90° C., 91° C., 92° C., 93° C., 94° C., 95° C., 96° C., 97° C., 98° C., 99° C., 100° C. or higher can be used. Incubation times can range on the order of seconds to minutes to hours.

Enzymatic fragmentation involves the use of nucleic acid cleavage or digestion enzymes. For example, a restriction enzyme or a nuclease. With regard to the kind of the restriction enzyme, it is also possible to use plural enzymes.

For mechanical crushing-based fragmentation, a method of cleaving the nucleic acid using balls of glass, stainless steel, zirconia, or the like can be used.

Generally, fragmentation of polynucleotide molecules by mechanical means (e.g., nebulization, sonication and Hydroshear methods) results in fragments with a heterogeneous mix of blunt and 3′- and 5′-overhanging ends. In some embodiments, it may be desirable to repair the fragment ends using methods or kits (such as the Lucigen DNA terminator End Repair Kit™) known in the art to generate ends that are optimal for insertion, for example, into blunt sites of cloning vectors. In a particular embodiment, the fragment ends of the population of nucleic acids are blunt ended. More particularly, the fragment ends are blunt ended and phosphorylated. The phosphate moiety can be introduced during an enzymatic treatment, for example using polynucleotide kinase.

Fragment sizes of the target nucleic acid can vary depending on the source target nucleic acid and the library construction methods used, but typically range from 50 to 600 nucleotides in length. In another embodiment, the fragments can be 200 to 700, 225 to 625, 315 to 525, 375 to 425, 400, 300 to 600, or, 200 to 2,000 nucleotides in length, or any range in between, inclusive. In another embodiment, the fragments can be 10-100, 50-100, 50-300, 100-200, 200-300, 50-400, 100-400, 200-400, 300-400, 400-500, 400-600, 500-600, 50-1000, 100-1000, 200-1000, 300-1000, 400-1000, 500-1000, 600-1000, 700-1000, 700-900, 700-800, 800-1000, 900-1000, 1500-2000, 1750-2000, and 50-2000 nucleotides in length.

### C. Fragmentation Simulation Method (FSM)

Although nucleic acid fragmentation methods are well known, the difficulty of controlling the random processes therein to generate nucleic acid fragments having a customized size is well known in the art and it has been determined herein that there is intrinsic variability in nucleic acid responses from a given sample to fragmentation. Accordingly, the present invention provides a fragmentation simulation method (FSM) to determine the parameters for a given nucleic acid fragmentation protocol necessary to achieve the customized size for a given master nucleic acid pool using aliquots of the master nucleic acid pool.

The method requires fragmenting at least two independent aliquots of the master pool of nucleic acid molecules in separate reactions, wherein the fragmentation conditions of each separate reaction are identical except for a single variable. For example, the incubation time can vary between aliquots that are fragmented using heat fragmentation wherein all other parameters of the heat fragmentation protocol are kept constant between processing of the aliquots. Depending on the fragmentation protocol and experimental design, time, temperature, pressure, shear force, reagent amount, reagent concentration, reagent activity, acoustic wavelength, acoustic frequency, or other parameter can vary while the remaining fragmentation protocol parameters remain constant. The aliquots can be processed simultaneously or sequentially, either alone or in groups. The number of aliquots can be from 2, 3, 4, 5, 6, 7, 8, 9, 10, 11, 12, 13, 14, 15, 16, 17, 18, 19, 20 or more and can include any range therein, inclusive.

The nucleic acid molecule fragment size distribution from each aliquot are then determined. This can be achieved in numerous ways well known to the skilled artisan (see, for example, Sambrook and Russell, Molecular Cloning, A Laboratory Manual, third edition). For example, a well-known technique for nucleic acid size distribution analysis uses nanopore technology to derive nucleic acid length distributions based on time of molecules occupying nanopores. Alternatively, size (i.e., length) separation based on electrophoretic mobility can be assessed using standard gel electrophoresis, capillary electrophoresis, and variations thereof, such as by combination with nanodrop spectrophotometry (Nanodrop Corp., USA). Visualization or computation algorithms can be used to analyze the observed fragment size distributions according to a number of metrics. For example, the mean, median, or mode of size lengths can be calculated to describe the fragment size distribution. As used herein, the term “mean” or “average” refers to the sum of nucleic acid sizes divided by the number of nucleic acid molecules. As used herein, the term “median” refers to the middle nucleic acid size when listing the sizes observed in numerical order. As used herein, the term “mode” refers to the nucleic acid size that occurs most often among the observed distribution of nucleic acid sizes. Practically, these measurements can be achieved by analyzing electropherogram or other representations of size distinguishing assays. For example, the “mean” can be calculated by taking the density of each band on an electropherogram and dividing by the total number of density-weighted bands. Similarly, the mode and median can be calculated according to methods described in the Examples. Alternatively, functional performance of the sample in an assay that is fragment size-sensitive, such as nucleic acid hybridization arrays, sequencing, or amplification assays, for example, derivative log ratio spread (dLRsd) values for array data quality, can be used to describe the fragment size distribution.

The resulting nucleic acid molecule fragment size distribution results are then plotted on a graph as a function of the value of the single variable for each aliquot. Carrying forward the heat fragmentation example, the fragment size distribution results would be plotted against the incubation time.

Once plotted, the data points are fitted to a curve to predict the value of the variable necessary to obtain a desired nucleic acid molecule fragment size distribution for the given sample.

Since the initial fragment size distribution of a given DNA sample can vary widely and because the rate of DNA fragmentation for each sample is also variable, the graph of a DNA sample's mode fragment size, f(t), as a function of the fragmentation time, t, may be modeled by more than a single equation. This is true for a single fragmentation method (e.g. thermodegradation), and across fragmentation methods including, but not limited to, enzymatic digestion, shearing, mechanical crushing, chemical treatment, nebulizing, and sonication. However, given continuous fragmentation (i.e., the fragmentation technology remains on, or any enzyme or chemical remains active), DNA fragment size will always be inversely proportional to fragmentation time and the slope of f(t) will always be less than zero. Therefore, the equation used to model DNA fragmentation in a given application may vary and more than a single equation may be used to approximate the same DNA fragmentation data. Specifically, the general form of the equations used to model DNA fragmentation may include a linear model (i.e., f(t)=−t), an exponential decay model (i.e., f(t)=θ₁e^{-tθ}^{₂}), an inverse power law (i.e.,

$$f ( t ) = \frac{1}{{θ}_{1} {t}^{{θ}_{2}}} ) ,$$

or others, where θ₁ and θ₂ are constant parameters used to obtain a curve that more closely models experimental data. Additionally, any number of parameters may be used to modify each of these general models or others in order to obtain functions that better approximate DNA fragmentation in the given application (e.g., inverse power law variants include, but are not limited to,

$$f ( t ) = \frac{1}{{t}^{{θ}_{1}}} , \\ f ( t ) = {θ}_{1} + \frac{1}{{θ}_{2} {t}^{{θ}_{3}}} , \\ f ( t ) = {θ}_{1} + \frac{{θ}_{2}}{{θ}_{3} {t}^{{θ}_{4}}} , \\ f ( t ) = {θ}_{1} + \frac{{θ}_{2}}{{( {θ}_{3} + {θ}_{4} t )}^{{θ}_{5}}} ,$$

and the like).

In some embodiments, an inverse power law function can be applied to the data to fit the curve since it has been determined herein that fragmentation size decay rates can be modeled using an inverse power law. For example, an inverse power law given by the mathematical formula,

$$f ( t ) = {θ}_{1} + \frac{{θ}_{2}}{{( t + {θ}_{3} )}^{{θ}_{4}}} ,$$

can be used to fit the curve of thermodegradation data, where f(t) is the mode DNA fragment size, t, is the single variable for each aliquot representing time of heat fragmentation, and θ₁, θ₂, θ₃, θ₄ are constant parameters unique for each DNA sample. The constant parameters, θ₁, θ₂, θ₃, θ₄, can be determined by performing an iterative regression, such as a least squares non-linear regression. Other methods for parametric regression analysis may also be used including, but not limited to, linear regression, simple regression, ordinary least squares, and polynomial regression. The skilled artisan will readily recognize that the type of analysis used will depend on the function used to model the data as well as the data itself.

Based upon the curve, the value of the single variable necessary to obtain the desired nucleic acid molecule fragment size distribution on the curve can be identified. This allows the skilled artisan to fragment the master pool of nucleic acid molecules or an aliquot thereof, wherein the fragmentation conditions are performed using the identified value of the single variable necessary to obtain the desired nucleic acid molecule fragment size distribution,

to thereby generate nucleic acid fragments having a customized fragment size distribution.

### D. Applications of the Fragmentation Simulation Method (FSM)

In some embodiments, the customized fragment size distribution for a given master pool of nucleic acid molecules may be determined based upon a particular intended use of the fragments that would benefit from having a defined input of nucleic acid molecule sizes. For example, many nucleic acid hybridization-based, sequencing-based, and/or amplification-based assays would benefit from nucleic acid inputs having a defined size.

Exemplary, non-limiting analytical techniques include Southern blotting, Northern blotting, comparative genomic hybridization (CGH), chromosomal microarray analysis (CMA), expression profiling, DNA microarray, high-density oligonucleotide microarray, whole-genome RNA expression array, polymerase chain reaction (PCR), digital PCR (dPCR), reverse transcription PCR, quantitative PCR (Q-PCR), single marker qPCR, real-time PCR, ligation chain reaction (sometimes referred to as oligonucleotide ligase amplification OLA), cycling probe technology (CPT), strand displacement assay (SDA), transcription mediated amplification (TMA), nucleic acid sequence based amplification (NASBA), rolling circle amplification (RCA) (for circularized fragments), invasive cleavage assays, nCounter Analysis (Nanostring technology), genome sequencing, de novo sequencing, pyrosequencing, polony sequencing, copy number variation (CNV) analysis sequencing, small nucleotide polymorphism (SNP) analysis, whole exome sequencing, in situ hybridization, either DNA or RNA fluorescent in situ hybridization (FISH), chromogenic in-situ hybridization (CISH), RNA sequencing, and epigenetic profiling, such as methylation pattern sequencing, phosphorylation pattern sequencing, and the like.

Included within the exemplary list are so-called “next-generation” sequencing techniques that may be amenable to performing large numbers of sequencing reactions in parallel and that would benefit from nucleic acid inputs having a defined size. Such techniques include pyrosequencing, nanopore sequencing, single base extension using reversible terminators, ligation-based sequencing, single molecule sequencing techniques, massively parallel signature sequencing (MPSS) and the like, as described in, for example, U.S. Pat. Nos. 7,057,056; 5,763,594; 6,613,513; 6,841,128: and 6,828,100; and PCT Published Application Nos. WO 07/121,489 A2 and WO 06/084132 A2.

Many of the technologies described in the exemplary list are also adapted for arrays, which are sensitive to size variations because a multitude of individual reactions occur in densely packed locations. As used herein, an “array,” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain.

Moreover, many of the technologies described in the exemplary list may further require additional nucleic acid processing steps in addition to nucleic acid fragmentation prior to performing an assay using the technology. In such cases, the FSM methods described herein can be adapted to incorporate these steps or model such steps if actual incorporation is prohibitive in order to more accurately predict the actual fragmentation kinetics that will result using the master pool or aliquot thereof. For example, modeling may be required where actual modification would interrupt the ability to accurately determine nucleic acid sizes. As used herein“nucleic acid modifying reaction” refers to a process step that directly or indirectly modifies a nucleic acid molecule. In one embodiment, the modification is direct. In another embodiment, the modification is indirect or could be indirect. The term includes not only nucleic acid fragmentation, but also any additional processing step that modifies or could modify a nucleic acid molecule in the protocol for a given intended use of the fragmented nucleic acids. In one embodiment, every nucleic acid modification step prior to application of the nucleic acid input into a desired assay is performed or modeled in the aliquots of the master nucleic acid pool in order to generate the FSM results. In another embodiment, every step prior to application of the nucleic acid input into a desired assay is performed or modeled in the aliquots of the master nucleic acid pool in order to generate the FSM results since such steps could modify the nucleic acids. In still another embodiment, every step suspected of modifying or being able to modify the nucleic acid input of a desired assay prior to application of the nucleic acid input in the assay is performed or modeled in the aliquots of the master nucleic acid pool in order to generate the FSM results. In yet another embodiment, at least one step rather than every step according to the different embodiments listed above is performed or modeled in the aliquots of the master nucleic acid pool in order to generate the FSM results. In another embodiment, the at least one or every nucleic acid modifying reaction or simulated reaction thereof can be performed before, simultaneously with, or after the fragmentation reaction.

For example, nucleic acid molecules or fragments thereof are typically labeled with a detectable label prior to performing the assay. Labeling means that a detectable substance is bound to a nucleic acid. The term “detectable label”” refers to any atom or moiety that can provide a detectable signal and which can be attached to a nucleic acid. Examples of such detectable labels include fluorescent moieties, chemiluminescent moieties, bioluminescent moieties, ligands, magnetic particles, enzymes, enzyme substrates, radioisotopes and chromophores. Accordingly, the detectable substance is not particularly limited and exemplary, non-limiting labeling agents include fluorescein isothiocyanate (FITC), Cy-dye (such as Cy-3 and Cy-5), Alexa, Green Fluorescent Protein (GFP), Blue Fluorescent Protein (BFP), Yellow Fluorescent Protein (YFP), Red Fluorescent Protein (RFP), Acridine, DAPI, Ethidium bromide, SYBR Green, Texas Red, rare-earth fluorescent labeling agent, TAMRA, ROX, digoxigein (DIG), biotin, and the like. As an example of utilizing biotin, when avidin is bound to biotin which has been bound to a probe, an alkaline phosphatase to which biotin has been bound is bound thereto, and nitroblue tetrazolium and 5-bromo-4-chloro-3-indolyl phosphate that are substrates for the alkaline phosphatase are added, purple coloration is observed and thus can be used for detection.

Moreover, labeling can be performed in a non-enzymatic manner. For example, the Universal Labeling System™ (ULS™) technology can be used (ULS™ array CGH Labeling Kit; manufactured by Kreatech Biotechnology BV Company) and the like can be also used. Briefly, ULS™ labeling is based on the stable binding properties of platinum (II) to nucleic acids (van Gijlswijk et al. (2001) Expert Rev. Mol. Diagn. 1:81-91). The ULS molecule consists of a monofunctional platinum complex coupled to a detectable molecule of choice. Alternative methods may be used for labeling the RNA, for example, as set out in Ausubel, et al. (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.).

As a method for fluorescent labeling, either labeling method of a direct labeling method and an indirect labeling method may be used. The direct labeling method means a method where a nucleic acid is transformed into a single-strand one, a short-chain nucleic acid is hybridized thereto, and a nucleotide compound to which a fluorescent substance (e.g., Cy-dye) has been bound is mixed with the nucleotide, thereby the nucleic acid is labeled in one step. The indirect labeling method means a method where a nucleic acid is transformed into a single-strand one, a short-chain nucleic acid is hybridized thereto, a nucleotide compound having a substituent capable of being bound to a fluorescent substance (e.g., Cy-dye), for example, a nucleotide compound having an aminoallyl group and the natural nucleotide are mixed together, a nucleic acid having the substituent is first synthesized, and then a fluorescent substance (e.g., Cy-dye) is bound through the aminoallyl group, thereby the nucleic acid being labeled.

As methods for introducing a labeling compound such as a fluorescent substance into the nucleic acid, a random primer method (primer extension method), a nick translation method, a PCR (Polymerase Chain Reaction) method, a terminal labeling method, and the like may be used.

The random primer method is a method where a random primer nucleic acid having several by (base pair) to over ten by is hybridized and amplification and labeling are simultaneously performed using a polymerase, thereby a labeled nucleic acid being synthesized. The nick translation method is a method where, for example, a double-strand nucleic acid to which nick has been introduced with DNase I is subjected to the action of a DNA polymerase to decompose DNA and simultaneously synthesize a labeled nucleic acid by the polymerase activity. The PCR method is a method where two kinds of primers are prepared and a PCR reaction is carried out using the primers, thereby amplification and labeling being simultaneously performed to obtain a labeled nucleic acid. The terminal labeling method is a method where, in a method of labeling a 5′-end, a labeling compound such as a fluorescent substance is incorporated into a 5′-end of a nucleic acid dephosphorylated with an alkaline phosphatase by a phosphorylation reaction with a T4 polynucleotide kinase. A method of labeling 3′-end is a method where a labeling compound such as a fluorescent substance is added to a 3′-end of a nucleic acid with a terminal transferase. As the labeled sample nucleic acid or the like, it is also possible to use an unpurified solution containing the same. In the case of using such an unpurified solution, an enzyme and the like still remain in the solution and hence, after preparation, it is preferable to deactivate the activity of the enzyme remaining in the solution. It is based on the viewpoint of preventing the influence on reproducibility of data. As methods for deactivating the enzyme, any methods may be possible as long as they can deactivate the enzyme but it is preferable to perform any one or both of a method of adding a chelating agent or a heating treatment at 60° C. or higher. The heating temperature is preferably 60° C. or higher, more preferably 63° C. or higher. The heating time is sufficiently 1 minute or more and most preferably, it is preferred to perform the heating treatment at 65° C. or higher for 5 minutes or more. Moreover, in the case of labeling method using a Klenow fragment, it is also possible to deactivate the activity of the enzyme using a vortex mixer or the like.

In some embodiments, modeling is required because actual nucleic acid modification is prohibitive. For example, some nucleic acid labeling strategies, as discussed further below, such as incorporation of Cy3 conjugates, Cy5 conjugates, or large moieties, affect nucleic acid electrophoretic mobility and thus size determination based on electrophoresis. The term “modeling” refers to mimicking the reaction conditions of the prohibitive treatment to the extent needed to avoid the prohibition. In the case of the nucleic acid labeling strategies, for example, the labeling reaction conditions, such as the protocol, salt, solvent, temperature conditions, and the like without including the prohibitive Cy3 or Cy5 conjugates.

Other nucleic acid modifying reactions are well known in the art and are routine for the nucleic acid assay technologies described herein. Exemplary, non-limiting examples of such reactions include in vitro transcription, amplification, methylation, demethylation, phosphorylation, dephosphorylation, linker addition or conjugation, nicking, ligation, blunting, digestion, and the like.

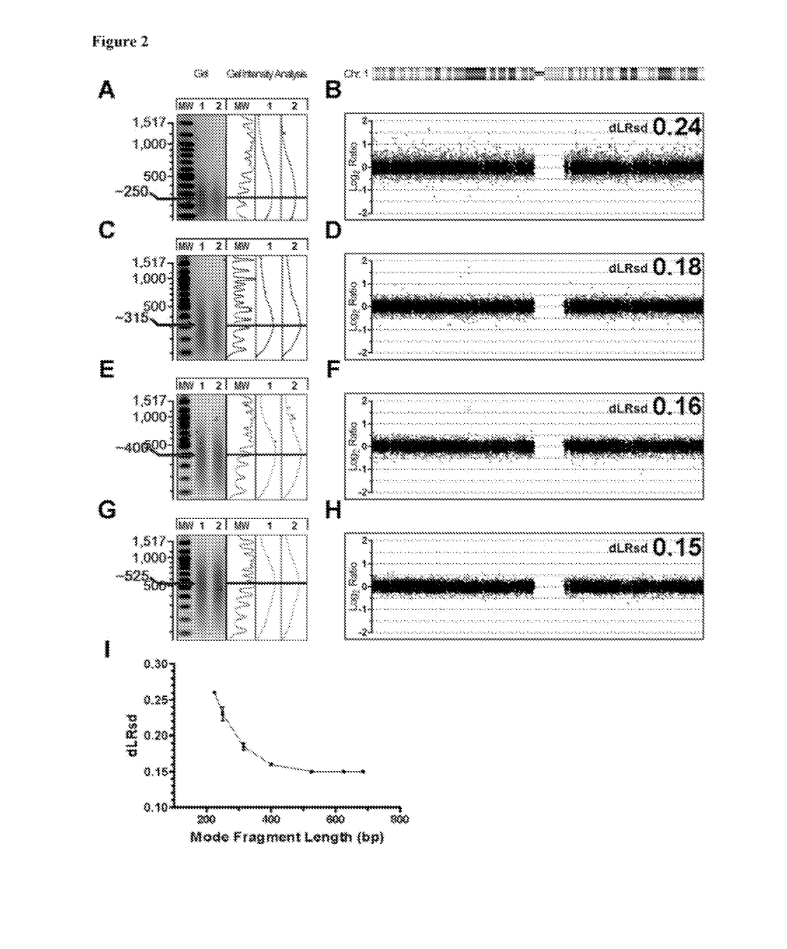

It has further been determined herein that having nucleic acid size-matched samples for competitive hybridization assays is an important factor, in addition to the individual nucleic acid size distributions for each sample, for improving data quality of competitive hybridization to a target sequence. For example, FIG. 2 demonstrates that competitive hybridization samples having sizes less than optimal for hybridization to a target sequence nevertheless produced high quality data in the assay when the samples were size-matched. As used herein, the term “competitive hybridization assay” refers to a technology requiring at least two samples containing nucleic acids that will compete with each other for binding to a target nucleic acid. Such assays are well-known in the art and include, for example, comparative genomic hybridization (CGH) and array-based comparative genomic hybridization (aCGH). The present invention further provides a method of generating nucleic acid fragments having customized and essentially identical fragment size distributions from each of at least two independent master pools of nucleic acid molecules to be fragmented comprising performing the methods described above using at least two master pools of nucleic acid molecules.

### EXEMPLIFICATION

This invention is further illustrated by the following examples, which should not be construed as limiting.

### Example 1

### Materials and Methods for Examples 2-7

### A. Tissue and Cell Line Specimens

Formalin-fixed paraffin embedded tissue specimens (n=122) and fresh-frozen tissue specimens (n=7) were obtained from six separate institutions under de-identified excess tissue protocols approved by institutional review boards at each institution (Boston Children's Hospital, Boston, Mass. (CHB); Brigham and Women's Hospital, Boston, Mass. (BWH); Children's Medical Center of Dallas, Dallas, Tex. (CMCD); Johns Hopkins Medical Institute, Baltimore, Md. (JHMI); Children's National Medical Center, Washington, D.C. (CNMC); and Marmara University Medical Center, Istanbul, Turkey (IST)). The IRB/ethics committee of each institution specifically waived the requirement for consent for these studies. All FFPE tissue specimens were human CNS malignancies or “normal” brain controls from non-neoplastic epilepsy specimens. Tumor samples were estimated to contain >50% tumor nuclei in all cases. Diagnoses were established by histologic examination according to the criteria of the World Health Organization classification by two neuropathologists (K.L.L. and S.S.). Primary glioma and other brain tumor cell lines were obtained either from the Dana-Farber Cancer Institute/Brigham and Women's Hospital Living Tissue Bank (DF/HCC) (n=64) or from the University of California San Francisco (UCSF) (n=7).

### B. Reference DNA

Commercial reference genomic DNA (created from fresh peripheral bloods pooled from five to seven healthy, karyotypically normal individuals) was purchased from Promega (cat. no. G1471/G1521, Madison, Wis.).

### C. DNA Extraction

1. FFPE Tissues

Genomic DNA was extracted from FFPE tissues using a protocol similar to that previously described in van Beers et al. (2006) Brit. J. Canc. 94:333-337. Briefly, 1 mm cores (two to five cores total) or 20 μm sections (three to five sections total) were taken from regions estimated to contain greater than 50% tumor cells based on previous pilot studies showing accurate detection of single copy gains and losses in samples with >40% tumor nuclei by pathologist estimate of hematoxylin and cosin (H&E) slides. Cores or sections were placed in sterile nuclease-free microcentrifuge tubes and paraffin was removed by treating the tissue in (1.2 ml) xylene. Samples were rinsed twice with 1.2 ml of 100% ethanol and allowed to dry at room temperature before the addition of 0.9 ml 1 M NaSCN and overnight incubation at 37° C. After 12-24 hrs, samples were rinsed twice in 0.9 ml 1×PBS. 0.34 ml of Buffer ATL (Qiagen, QIAamp DNA FFPE Tissue Kit cat. no. 56404, Valencia, Calif.) and 40 μl of Proteinase K (20 mg/mL) (Qiagen, cat. no. 19131) were added and samples were incubated in a thermomixer (Eppendorf, cat. no. 022670000, Hamburg, Germany) set at 56-58° C. and 450 rpm. An additional 40 μl Proteinase K was added every 8-12 hrs for a period of 48-72 hrs. Samples were allowed to cool to room temperature before the addition of 10-20 μl RNase A (100 mg/mL) (Qiagen, cat. no. 19101) and a 5-10 minute incubation at room temperature. After adding 400 μl of Buffer AL (Qiagen QIAamp DNA FFPE Tissue Kit), samples were placed in thermomixer at 60° C. for 10 minutes. 440 μl of 100% ethanol was added and each sample was split between two QIAamp MinElute Columns (Qiagen QIAamp DNA FFPE Tissue Kit). Following successive washes with 500 μl Buffer AWI (Qiagen QIAamp DNA FFPE Tissue Kit) and 500 μl 80% ethanol, DNA was eluted in 50-100 μl H₂O.

2. Frozen Tissues and Cells

Genomic DNA was extracted from frozen tissue and cell line samples using the DNeasy Blood & Tissue Kit (Qiagen, cat. no. 69504). The manufacturer's protocol was utilized with the inclusion of the optional RNase A treatment and the replacement of Buffer AW2 with 80% ethanol. DNA was eluted in 100-200 μl H₂O.

3. Fragmentation Simulation Method (FSM) Analysis

Prior to FSM analysis, all DNA samples were concentrated using 30 K MWCO Amicon Ultra Centrifugal Filter Units (Millipore, cat. no. UFC503096, Billerica, Mass.). The use of these filters also removes ssDNA and dsDNA fragments of 50-60 nt in length, and facilitates the serial dilution of residual salt and/or solvent in the purified DNA samples. Concentrated DNA samples were quantified by absorbance spectroscopy with a NanoDrop 1000 (Thermo Fisher) and diluted to working concentrations specific to Agilent aCGH array-dependent requirements (e.g. 125 ng/μL for 1 M arrays or 62.5 ng/μL for 180 K arrays). Briefly, a minimum of 240 ng DNA was removed from each sample and brought to a total volume of 32 μl with H₂O. This solution was then split into 8 μl aliquots in the same 200 μl PCR tubes that were to be used for the Universal Linkage System™ (ULS; Kreatech Diagnostics and Agilent Technologies) labeling reactions. These four aliquots were heat-fragmented at 95° C. in a PCR thermocycler for either 0, 0.5, 1, or 2 minutes (FFPE samples) or 0, 2, 4, or 6 minutes (frozen tissue/cells) immediately followed by a 4° C. cycle of at least 4 minutes duration. Using volume and composition proportions consistent with the 1 M array ULS labeling reaction, 2 μl of ULS labeling simulation solution (50% 10× Labeling Solution (Agilent Technologies, Genomic DNA ULS Labeling Kit cat. no. 5190-0419, Santa Clara, Calif.), 25% 20 mM NaCl, 25% DMF) was then added to each of the four aliquots before simulated ULS labeling reaction conditions were initiated (30 min at 85° C. then ≧10 min at 4° C. in PCR thermocycler). Sample aliquots were combined with 4 μl Orange (6×) Gel Loading Dye (New England Biolabs, cat. no. B7022S, Ipswich, Mass.) and loaded on 1.5% agarose 1×TBE gels prior to electrophoresis at 100-120 V. Gels were stained with GelRed Nucleic Acid Stain (Phenix Research Products, cat. no. RGB-4103, Candler, N.C.). Utilizing open-source ImageJ analysis software (U.S. National Institutes of Health, Bethesda, Md.), the mode fragment size of each aliquot was approximated by referencing the maximum intensity of each smear with the bands of a 100 bp DNA Ladder (New England Biolabs, cat no. N3231S). The fragmentation of each sample was modeled using this data in combination with Equation 1

$$( f ( t ) = {θ}_{1} + \frac{{θ}_{2}}{{( t + {θ}_{3} )}^{{θ}_{4}}} )$$

and JMP 8 analysis software (SAS Institute Inc., Cary, N.C.), and an optimal heat fragmentation time was determined.
D. Array Comparative Genomic Hybridization (aCGH)

1. FSM ULS

Purified DNA extracts from FFPE tissues, frozen tissues, and frozen cells were heat fragmented as indicated by FSM analysis. Subsequently, ULS labeling (Agilent Technologies, Genomic DNA ULS Labeling Kit cat. no. 5190-0419. Santa Clara, Calif.) was performed according to the manufacturer's suggested protocol. Briefly, 2 μg DNA from each sample was combined with 2 μL ULS-Cy5 Reagent (Genomic DNA ULS Labeling Kit) and 2 μL 10× Labeling Solution (Genomic DNA ULS Labeling Kit) prior to 30 min at 85° C. and ≧10 min at 4° C. in a PCR thermocycler. An equal mass of either male or female reference DNA was heat-fragmented according to FSM predictions and then labeled with the ULS-Cy3 Reagent (Genomic DNA ULS Labeling Kit). Unincorporated dye was removed using Genomic DNA Purification Modules (Agilent Technologies, cat. no. 5190-0418). The entire volumes of the Cy5-labeled sample DNA and the Cy3-labeled reference DNA were combined together with 37.8 μL H₂O, 50 μL Cot-I DNA (Invitrogen, cat. no. 15279-011, Carlsbad, Calif.), 5.2 μL 100× Blocking Agent (Agilent Technologies, Oligo aCGH Hybridization Kit cat. no. 5188-5220), and 260 μL 2× Hi-RPM Hybridization Buffer (Agilent Technologies, cat. no. 5190-0403) before denaturation (3 min at 95° C.) and pre-hybridization (30 min at 37° C.). 130 μL Agilent-CGH block (Agilent Technologies, cat. no. 5190-0421) was added to each hybridization solution before 490 μL of the combined solution was applied to a gasket slide (Agilent Technologies, cat. no. G2534-60003). A 1×1 M SurePrint G3 Human CGH Microarray (Agilent Technologies, cat. no. G4447A) was paired with each gasket slide in a SureHyb Enabled Hybridization Chamber (Agilent Technologies, cat. no. G2534A) and the differentially labeled DNA samples were hybridized (65° C.) to the microarray for 40-72 hrs in a hybridization oven (Agilent Technologies, cat. no. G2545A). During hybridization the slides were rotated at 19 rpm.

2. Standard ULS

DNA extracted from FFPE tissues was not subjected to additional fragmentation prior to ULS labeling. The intact DNA extracted from frozen tissues and cells, as well as reference DNA samples, were heat fragmented for ten minutes as suggested by the manufacturer's standard ULS protocol. The remainder of both labeling and hybridization procedures was identical to those of the FSM ULS method.

3. Self-Hybridizations

Single sample self-hybridizations utilized male reference genomic DNA (Promega, G1471, Madison, Wis.). DNA was suspended in nuclease-free H₂O using 30 K MWCO Amicon Ultra Centrifugal Filter Units. 500 ng aliquots were heat-fragmented (95° C.) for varying lengths of time and then differentially labeled with ULS-Cy3 Reagent and ULS-Cy5 Reagent before hybridization to 4×180 K SurePrint G3 Human CGH Microarrays (Agilent Technologies, cat. no. G4449A), and according to the manufacturer's standard ULS protocol.

4. Microarray Washing, Scanning, and Feature Extraction

Microarrays and gaskets were disassembled at room temperature in Wash Buffer 1 (Agilent Technologies, cat. no. 5188-5221) and quickly moved to a second dish containing Wash Buffer 1 and a stir bar rotating at speed sufficient for gentle agitation of the liquid's surface. After 5-30 minutes, slides were moved to a dish containing Wash Buffer 2 (Agilent Technologies, cat. no. 5188-5222) and a stir bar and agitated at 37° C. for 1 minute. Slides were then washed in anhydrous acetonitrile (Sigma-Aldrich, cat. no. 271004, St. Louis, Mo.) for 10-15 sec before being removed and placed in a slide holder (Agilent Technologies, cat. no. G2505-60525) with an Ozone-Barrier Slide Cover (Agilent Technologies, cat. no. G2505-60550). Microarrays were scanned immediately with a DNA Microarray Scanner (Agilent Technologies, cat. no. G2505C) at 3 μm resolution. Scanned images were processed using Agilent Feature Extraction v10.7 and FE Protocol CGH_{—}107_Sep09. Quality control dLRsd statistics were recorded as reported in the QC Metrics file generated by the software.

### E. Data Analysis

Copy number analysis was performed using the DNA Analytics module of Agilent Genomic Workbench 6.5. Log₂ ratios were corrected for a periodic “wave” artifact that correlates with GC content using the software's GC correction tool with a GC window size of 2 kb. The ADM-2 algorithm was used with a threshold of 6.0 to detect significantly aberrant genomic regions and detected regions were filtered for those spanning more than five probes (˜10 kb) with an average absolute log₂ ratio >0.3. Array data has been published in compliance with MIAME 2.0 guidelines and deposited in the publicly available ArrayExpress database.

### Example 2

### Array Performance is Improved when Test and Reference DNA Samples Possess Similar Fragment Sizes

The effects of DNA fragment size on aCGH data quality were determined. To do this, a series of self-hybridizations were conducted using a commercially available, high-quality genomic DNA (gDNA) sample that is a common reference standard in Agilent aCGH analyses (Promega, G1471, Madison, Wis.). The reference gDNA had a high molecular weight distribution at the outset (mode fragment length >10 kb). The sample was split into eight identical aliquots and heat-fragmented at 95° C. for either 0, 5, or 10 minutes to generate a distribution of DNA sizes. The resulting DNA fragments demonstrated modes of 525, 225, and 140 bp. Each aliquot was labeled separately and paired in four combinations to create both size-matched and mismatched fragment pairs (matched pair: 225/225 and mismatched pairs: 525/225, 525/140, 225/140). These paired samples were then hybridized to Agilent 180 K feature arrays to model the variation in DNA fragment size commonly present in test and reference samples competitively hybridized to arrays.

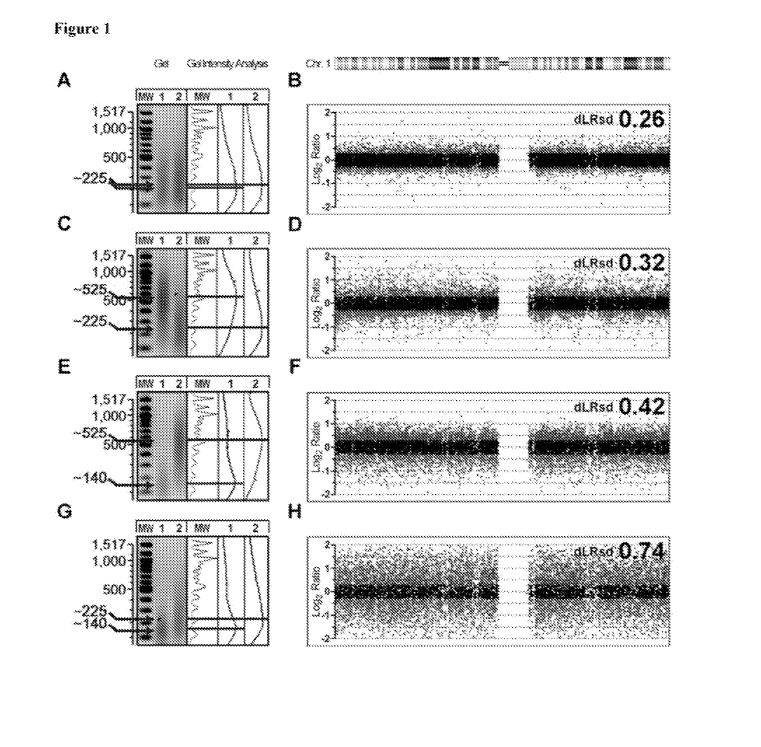

Despite the initially intact and identical condition of the gDNA in each pair, three out of four self-hybridizations failed to achieve derivative log ratio spread (dLRsd) values less than 0.3, a primary QC metric and threshold for array data quality (FIG. 1; Hostetter et al. (2010) Nucl. Acids Res. 38:e9; and Pinto et al. (2011) Nat. Biotech. 29:512-520). The self-hybridization pair with matched DNA size distributions that had been exposed to identical fragmentation conditions resulted in a dLRsd of less than 0.3 (FIGS. 1A-1B), indicating a hybridization likely to yield robust copy number data. The introduction of even moderate size mismatches (300 bp differential) was sufficient to introduce profound changes in final data quality, even when the mismatch resulted from an increase in fragment size (FIGS. 1C-1D). Additional loss of data quality was noted when the difference in fragment sizes between the competitively hybridized DNA samples was further increased to 385 bp (FIGS. 1E-1F). The magnitude of the size mismatch effect on data quality is not completely dependent on the magnitude of the size differential, however; as seen in the high dLRsd of the array data in FIGS. 1G-1H, it is likely that decreased fragment size also adds complexity to the mechanism. These findings demonstrate that fragment size matching is critical for reducing the variability of array data quality even when using highly intact, optimal DNA samples.

### Example 3

### Determination of Optimal Mode Fragment Size in Size-Matched Samples

Prior studies have indicated that experimental samples with fragment size distributions less than 300 bp may be a source of inconsistent aCGH performance (van Beers et al. (2006) Brit. J. Canc. 94:333-337; Johnson et al. (2006) Lab. Invest. 86:968-978; Hostetter et al. (2010) Nucl. Acids Res. 38:e9; and Alers er al. (1999) Genes, Chrom. Canc. 25:301-305). Given that matching fragment and reference DNA sizes improves results and might alter baseline performance, a determination of the optimal fragment size under size-matched conditions was re-evaluated. To test this, additional self-hybridizations were performed using the reference gDNA sample and generated a spectrum of size distributions by varying heat fragmentation times. In total, 16 size-matched self-hybridizations representing seven unique size distributions (range≈200-700 bp; mode fragment lengths≈225, 250, 315, 400, 525, 625, and 680 bp), were measured in duplicate (n=5) or triplicate (n=2). In contrast to the size mismatched pairs shown in FIGS. 1C-1H, all self-hybridizations between samples with matched fragment sizes yielded data within the acceptable range (dLRsd<0.3), regardless of length of the DNA fragments (FIG. 2). However, a significant correlation between decreased dLRsd and increased mode fragment size (r=−0.85, p=0.015) (FIG. 2) was observed with optimal data quality achieved at mode fragment sizes greater than 400 bp (FIG. 2I). Overall, it was observed that optimal aCGH data quality is produced with DNA fragment distributions of paired samples of similar sizes and mode fragment size greater than or equal to 400 bp.

### Example 4

### Tissue Sample DNA Responses to Heat Fragmentation Conditions are Intrinsically Variable and Must be Determined Empirically

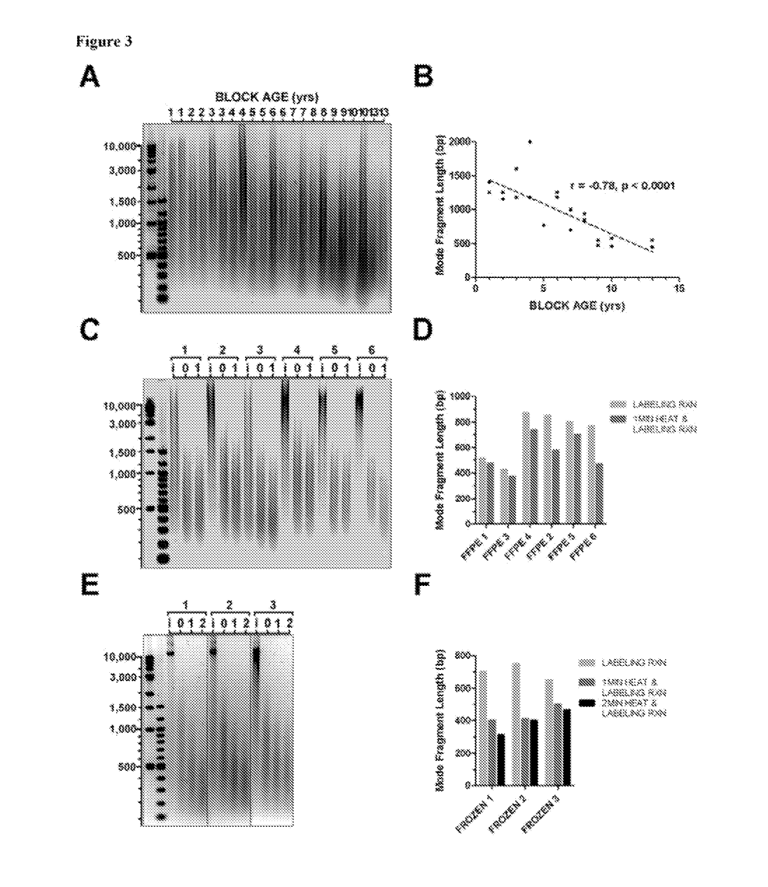

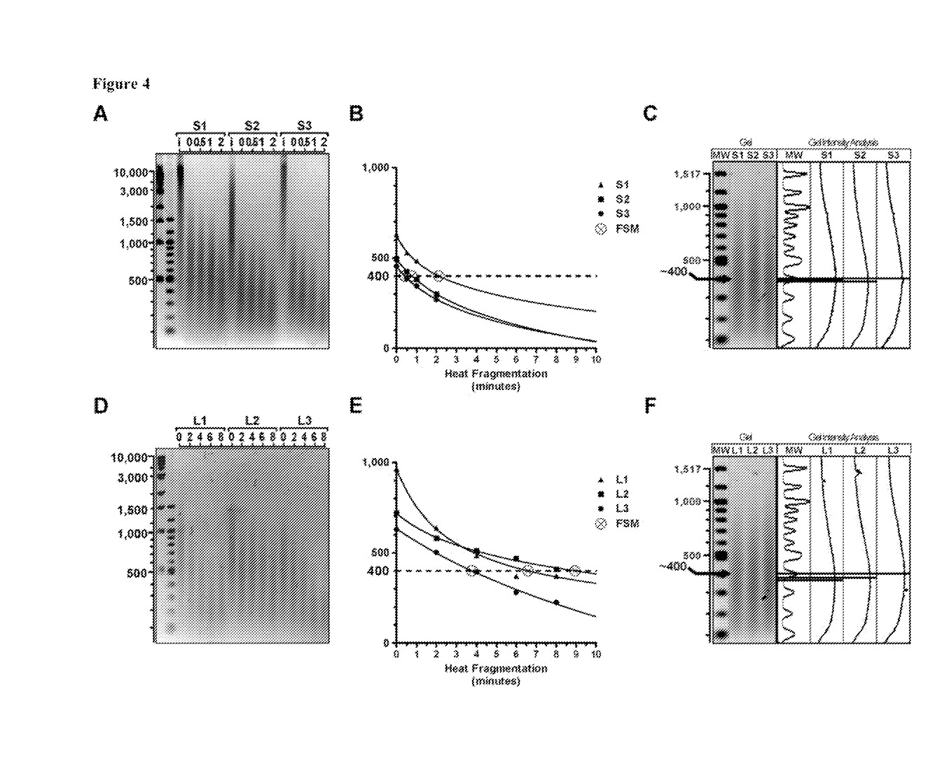

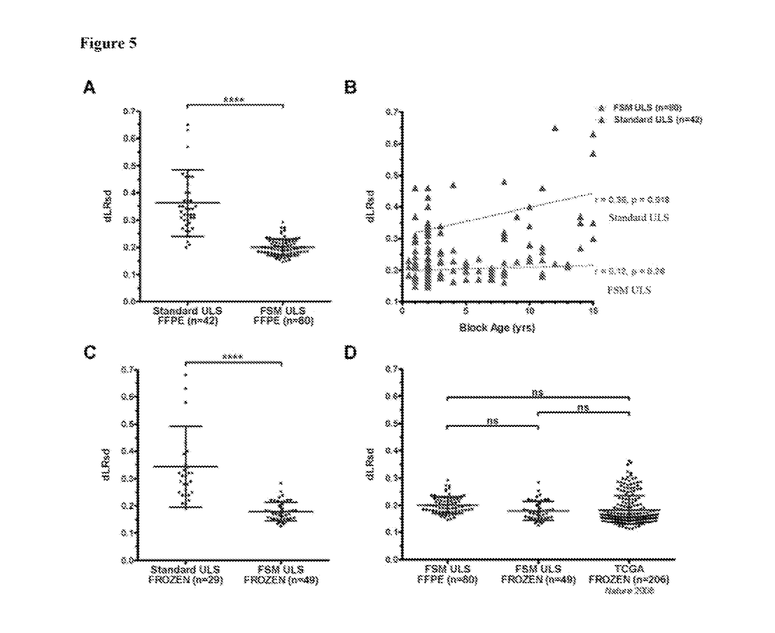

Utilizing the DNA extraction protocol with over 100 FFPE brain tumor specimens (block ages ranging from one to 15 years, all estimated to contain >50% tumor tissue) obtained from six different institutions, 100% of samples yielded DNA with average fragment sizes greater than 400 bp. Indeed, for most samples the fragment sizes were well above this size threshold and in agreement with general size ranges reported in other studies (van Beers et al. (2006) Brit. J. Canc. 94:333-337; and Hostetter et al. (2010) Nucl. Acids Res. 38:e9). Agarose gel electrophoresis of 22 DNA extracts from FFPE tissue blocks ranging in age from one to 13 years confirmed this observation (FIG. 3A). In fact, plotting the mode fragment size of each smear against block age revealed a statistically significant relationship (r=−0.77, p<0.0001) between advanced age and decreased fragment size (FIG. 3B). Despite this relationship, the results support the conclusion that the initial (post-extraction) degradation of FFPE-derived DNA does not preclude obtaining fragment distributions within the optimal range (FIG. 2), even among DNA samples isolated from archival specimens over ten years old.

Previous mechanistic studies of DNA thermodegradation describe significantly different rates of depurination and subsequent fragmentation in single versus double-stranded DNA (Lindahl (1993) Nature 362:709-715; and Suzuki et al. (1994) Nucl. Acid Res. 22:4997-5003) and other studies have exposed the commonly overlooked role of nucleic acid degradation in standard PCR conditions (Alers et al. (1999) Genes, Chrom. Canc. 25:301-305; and Gustafson et al. (1993) Gene 123:241-244). In light of these studies, whether the thermodegradation that occurs during labeling and other standard aCGH steps contributed to the variability in the aCGH results was sought to be determined. Since the ULS Cy5 and Cy3 conjugates affect the electrophoretic mobility of DNA, a simulated labeling reaction that exactly mimics the salt, solvent, and temperature conditions of the ULS labeling reaction was designed. DNA samples were then assessed by gel electrophoresis following these simulated labeling conditions. Measured as the change in mode fragment size following heat fragmentation and/or labeling conditions, significantly variable rates of thermodegradation was observed across samples (FIGS. 3C-3F), despite reproducibility in any given sample. Additionally, variable thermodegradation rates were observed even among samples of apparently similar initial size distribution, which confounded attempts to reliably predict the ultimate fragment size distribution of any given sample after heat fragmentation and labeling procedures based on the initial fragment size distribution of that sample. This intrinsic variability in DNA response to heat conditions in aCGH procedures was seen in all types of specimens, including fresh, frozen, and FFPE specimens alike (FIGS. 3C-3F).

### Example 5

### Application of the Fragmentation Simulation Method (FSM) Allows Reliable Control of DNA Fragmentation Distributions and Improves Quality of aCGH Results

The variability observed in DNA thermodegradation rates suggested that the predefined fragmentation conditions used in published aCGH-FFPE protocols were unlikely to achieve the size uniformity required for optimal aCGH results. To increase the number of samples that yield high-quality aCGH data, a Fragmentation Simulation Method (FSM) was developed that allows fragmentation conditions to be tailored to individual samples using a single, standardized protocol. Observation of the time course of DNA thermodegradation in both fresh/frozen and FFPE DNA samples suggested that fragment size decay rates might best be modeled using an inverse power law as follows:

$$f ( t ) = {θ}_{1} + \frac{{θ}_{2}}{{( t + {θ}_{3} )}^{{θ}_{4}}} ,$$

where f(t) is the mode DNA fragment size, in base pairs, of a sample's fragment distribution immediately prior to hybridization (after a variable time of heat fragmentation and a simulated labeling reaction), t is time of heat fragmentation in minutes, while θ₁, θ₂, θ₃, and θ₄ are constant parameters unique for each sample. Data points (n≧4) were experimentally determined by exposing aliquots of a DNA sample (≧50 ng each) to variable times of heat fragmentation (e.g. t=0, 0.5, 1, and 2 minutes), followed by a simulated labeling reaction. The aliquots were then subjected to agarose gel electrophoresis and the open source ImageJ analysis software was used to determine the mode fragment size of each aliquot's fragment distribution, f(t) (FIGS. 4A and 4D). An iterative least squares non-linear regression was then used to derive parameter values (θ₁, θ₂, θ₃, and θ₄) and fit a curve to the experimentally observed thermodegradation for each sample.

Once these parameters were determined, the completed model was used to predict the amount of heat fragmentation time, t, required to achieve an optimal mode fragment size, f(t), in each DNA sample (FIGS. 4B and 4E). Analysis of test samples subjected to heat fragmentation for a length of time indicated by the FSM and subjected to ULS labeling showed that the desired target fragment size distribution was attained (FIGS. 4C and 4F). Following the FSM and ULS labeling, samples are hybridized to arrays without further modification. Thus, FSM provides a single, standardized protocol that accommodates the unique variation in the fragment size of an input DNA sample and its inherent thermodegradation rate.

### Example 6

### FSM Improves aCGH Quality and Reduces Sample-To-Sample Variability in FFPE Samples

To determine whether the FSM method might improve the results obtained from both FFPE and non-FFPE tissue samples, array data obtained using the FSM protocol were rigorously compared with data obtained using the standard manufacturer's ULS protocol. Hybridizations were performed using Agilent SurePrint stock arrays with a 1 million feature resolution. A diverse set of FFPE tumor specimens (n=122), frozen tumor tissues (n=7), primary tumorspheres and other tumor cell cultures (n=71) were analyzed (Table 1). First, differences in the data quality generated by FFPE central nervous system (CNS) malignancies obtained from multiple institutions from blocks of various ages (one to 15 years) were assessed. The quality of the array data processed according to the standard ULS protocol (n=42, μ_{dLRsd}=0.36, σ_{dLRsd}=0.12) was inferior to that of samples processed according to the FSM ULS protocol (n=80, μ_{dLrsd}=0.20, σ_{dLRsd}=0.03) with the difference reaching statistical significance (p<0.0001) as assessed by t and F tests (FIG. 5A).

Noting significantly less variance in the quality of the FSM ULS subset, whether the age of the tissue blocks and the resultant array quality are indeed related to one another, as previously suggested, was tested. In the standard ULS set, the correlation between increased sample age and lowered dLRsd was strong and significant (r=0.36, p=0.018), however this was not observed in the FSM ULS subset (r=0.12, p=0.26) (FIG. 5B).

Since an optimal clinical laboratory protocol would ideally be the same for either fresh or fixed tissues and also because the ULS direct labeling approach has practical and experimental advantages over the commonly used enzymatic methods (Alers et al. (1999) Genes, Chrom. Canc. 25:301-305), the utility of the FSM ULS protocol using DNA isolated from either frozen tissue (n=7) or frozen cells (n=71) and Agilent 1 M feature arrays was examined. As observed in the FFPE sample sets, the subset of frozen samples processed with the FSM ULS protocol (n=49, μ_{sLRsd}=0.18, σ_{dLRsd}=0.04) demonstrated significantly (p<0.0001) higher quality and less variance than those processed according to the standard ULS protocol (n=29, μ_{dLRsd}=0.34, σ_{dLRsd}=0.15) (FIG. 5C). Finally, quality was compared across all of the FFPE and frozen sample sets as well as a previously published set of Agilent 244 k array data generated by The Cancer Genome Atlas project (TCGA) using fresh-frozen glioblastoma tissue specimens and traditional enzymatic DNA labeling (n=206, μ_{dLRsd}=0.18, σ_{dLRsd}=0.05) (Network TCGAR (2008) Nature 455:1061-1068). One-way ANOVA and Tukey's multiple comparison test revealed significant differences between the standard ULS subsets and each FSM subset as well as the TCGA subset (p<0.001). As depicted in FIG. 5D, no significant difference was measured, however, between the FSM ULS FFPE subset, the FSM ULS frozen tissue subset, and the TCGA frozen tissue subset (p>0.05). Importantly, FIG. 5 demonstrates that the FSM method enables the use of both fresh/frozen and fixed tissue sources for similarly robust, high-resolution aCGH data.

### Example 7

### DNA Fragment Size Matching Facilitated by the FSM Method is More Critical to Array Quality than Previously Identified Factors

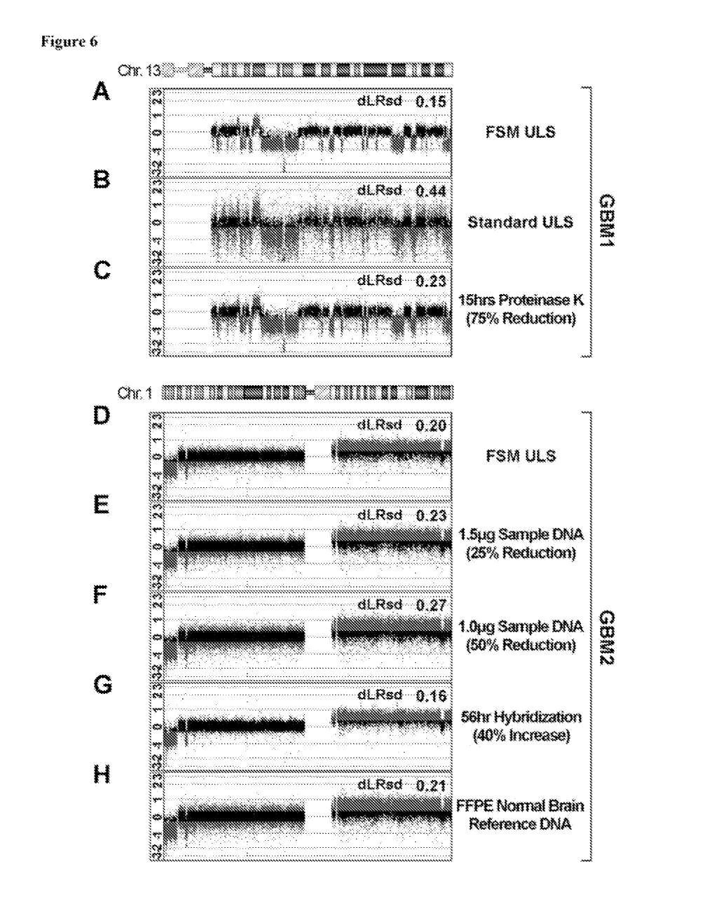

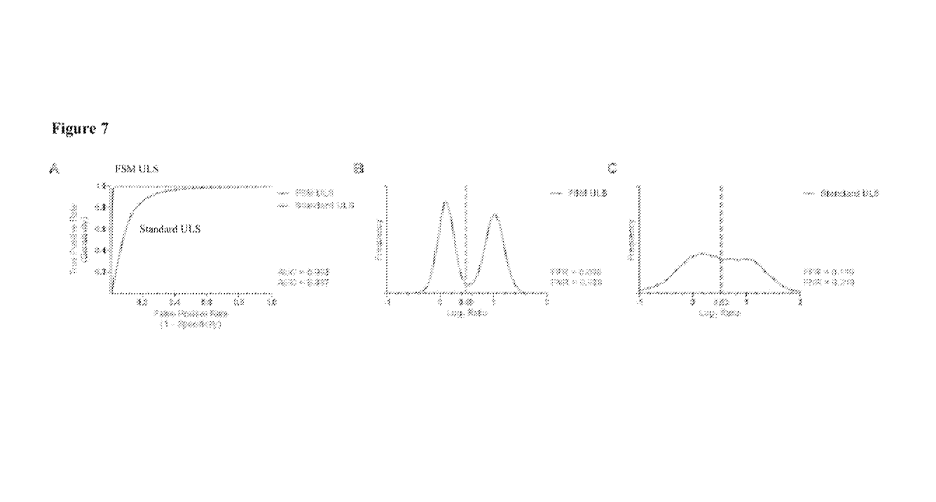

Having demonstrated the highly significant contributions of FSM analysis and matched DNA fragment sizes to aCGH quality, the relative effects of fragment size compared to other previously reported variables such as Proteinase K digestion time, array hybridization time, and concentration and source of DNA in array hybridization reactions were assessed. DNA from a single FFPE tumor specimen, GBM1 (characterized by complex and highly aberrant copy number changes involving single-copy gains, single-copy losses, and regions of homozygous deletion on chromosome 13), was processed under multiple conditions and assayed with Agilent 1 M feature arrays. A comparison of FIGS. 6A and 6B supports the previous assertions regarding the significant improvement of data quality enabled by the FSM. Compared with data obtained following the FSM ULS protocol (FIG. 6A) the standard ULS protocol yielded a higher dLRsd value (0.44) (FIG. 6B) that precluded accurate detection of copy number aberrations (FIG. 7).

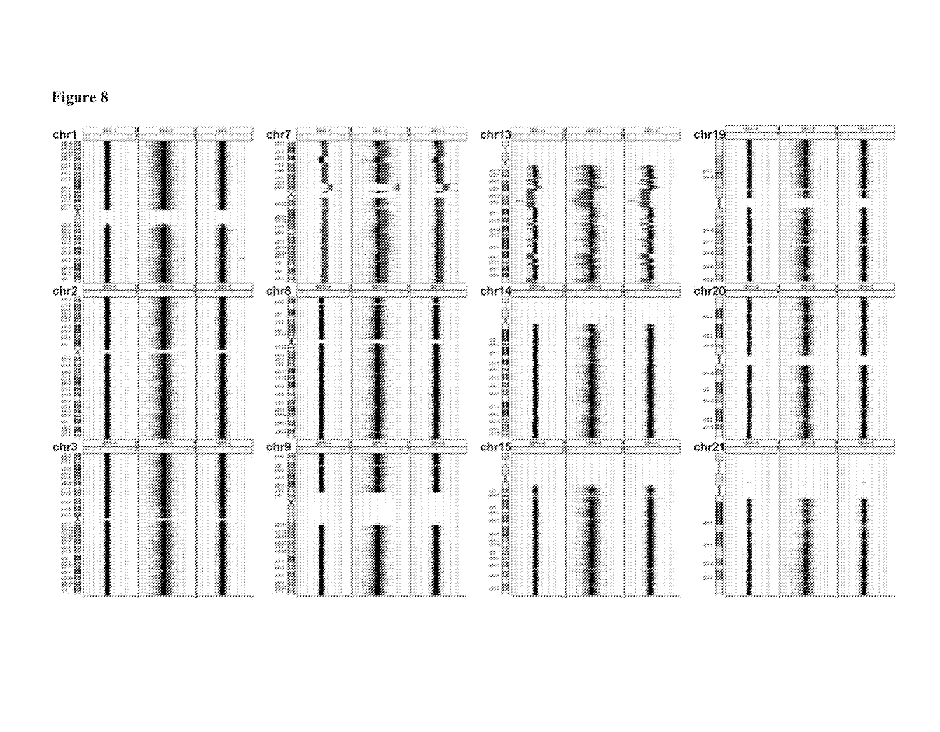

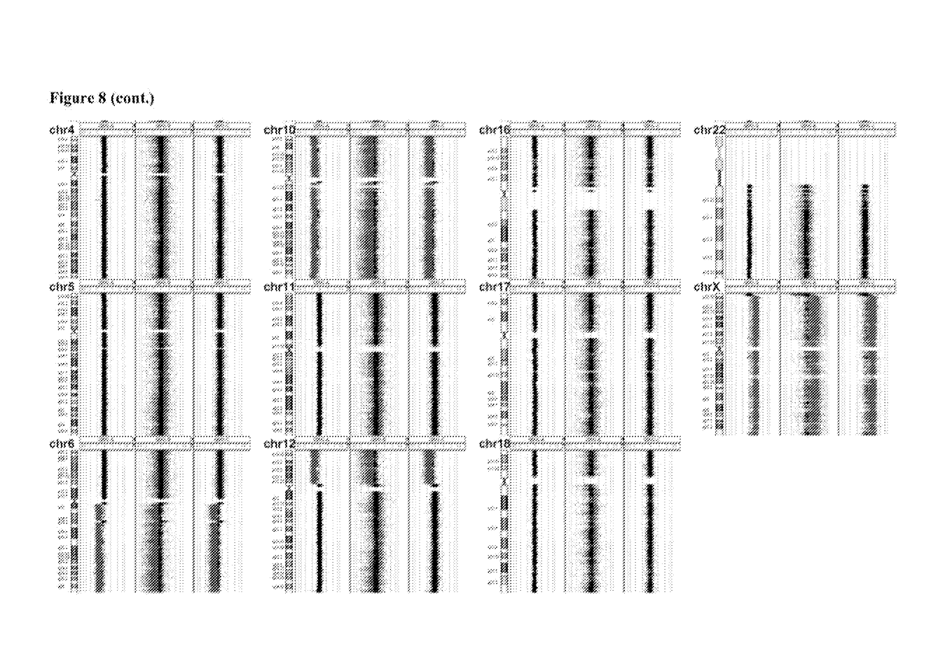

The duration of Proteinase K digestion during DNA extraction has frequently been identified as playing a critical role in the liberation of DNA from DNA-protein crosslinks and, consequently, it is thought to play a role in DNA labeling efficiency, hybridization, and resulting aCGH quality (Paris et al. (2007) The Prostate 67:1447-1455; van Beers et al. (2006) Brit. J. Canc. 94:333-337; Wessels et al. (2002) Canc. Res. 62:7110-7117; Alers et al. (1997) Lab. Invest. 77:437-448; van Gijlswijk et al. (2001) Exp. Rev. Mol. Diagnost. 1:81-91; and Hostetter et al. (2010) Nucl. Acids Res. 38:e9). The Agilent 1 M array data shown in FIG. 6C was produced from GBM1 DNA exposed to only 15 hours of Proteinase K digestion rather than the 64) hour digestion in the typical FSM ULS protocol used for the data in FIG. 6A. The sample was otherwise processed according to an identical FSM ULS protocol. The effect of the reduced Proteinase K digestion was measurable by dLRsd (Δ_{dLRds}=0.08), although the data quality (dLRsd=0.23) was well within recommended QC guidelines (dLRsd≦0.30) and aberrations across the whole genome were readily identified visually (FIG. 8) and algorithmically. The chromosome 1 data shown in FIGS. 6D-6H were generated using a single DNA sample from FFPE specimen, GBM2, and arrayed using five Agilent 1 M arrays. The data shown in FIG. 6D represents baseline conditions (FSM ULS protocol, 2 μg each of GBM2 and Promega reference DNA, 40 hr hybridization). Single conditions were varied to generate the data shown in FIGS. 6E-6H.

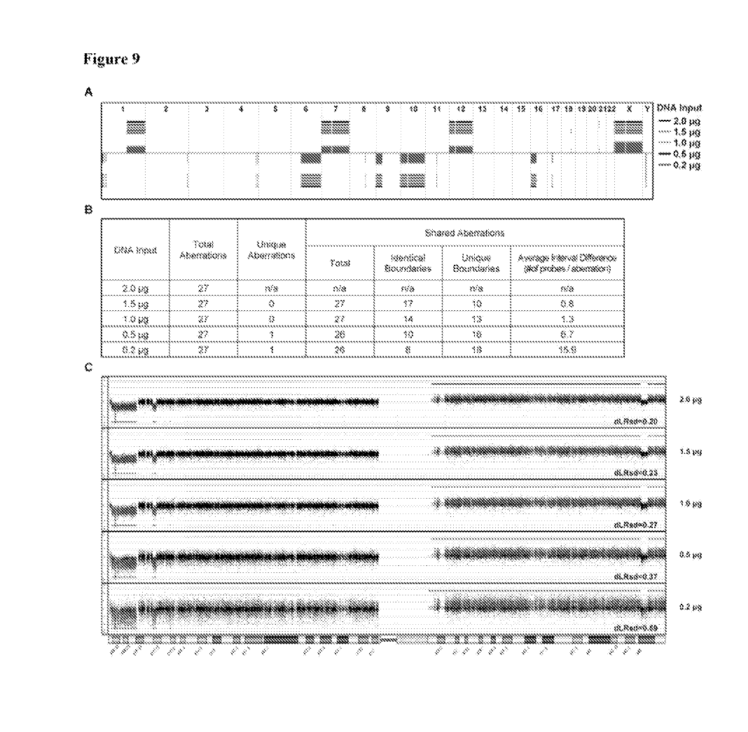

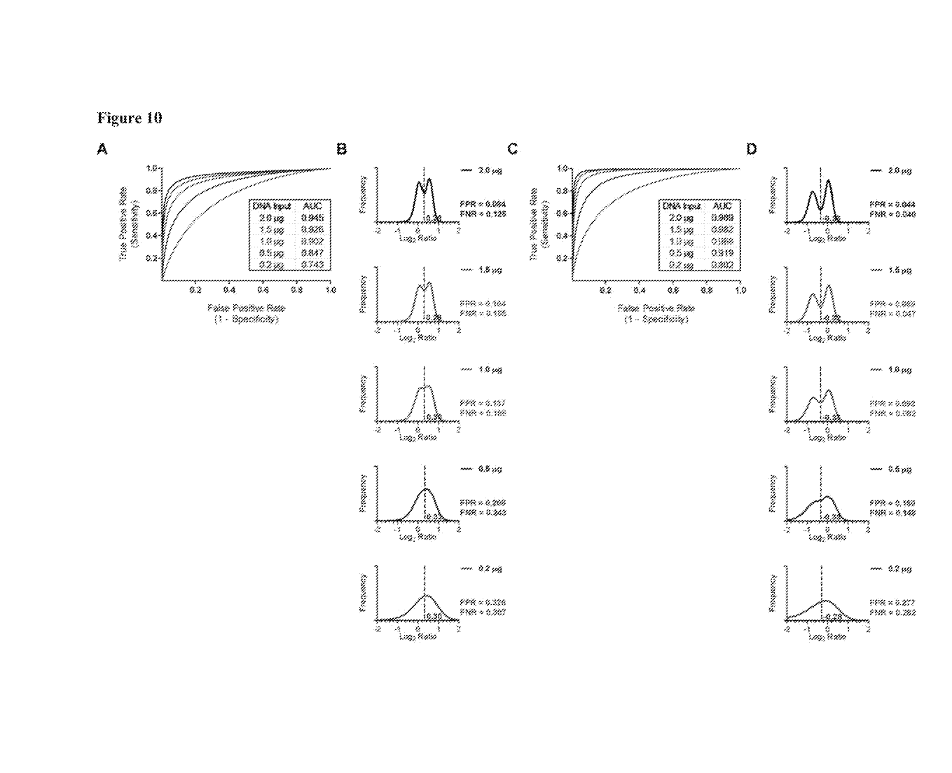

The tissue requirements of the assay are a critical factor and, as such, whether the FSM method would allow input of less DNA and still be able to generate robust results was sought to be determined. The resultant data from Agilent 1 M array hybridizations with 25% and 50% reductions of DNA input (both tissue DNA and reference DNA) relative to the standard DNA input are shown in FIGS. 6E-6F, respectively (data from additional hybridizations with 75% and 90% reductions of DNA input provided in FIG. 9, DNA input ranging from 0.2-2.0 ug). While the expected negative trend was observed in the data quality of these arrays, it is to be noted that even the dLRsd of the array hybridized with 1 ug DNA input (50% lower than standard) was still within an acceptable range (0.27). Perhaps more importantly, detection of copy number alterations by calling algorithms was 100% concordant with that of the baseline data shown in FIG. 6D (concordance was measured as proportion of total aberrations detected with overlapping genomic position). This held true even on detailed copy number analysis of over 27 tumor specific aberrations (FIG. 9). Examination of probe level sensitivity and specificity data for single copy gain/loss also showed highly reliable false positive/negative rates (FPR, FNR<0.20) at 1 ug of input DNA and reasonable performance even when only 0.2 ug of DNA was utilized (FIG. 10).

Increased duration of hybridization is thought to positively impact the quality of array data and, because hybridization beyond 40 hrs may be of practical benefit in many clinical laboratory settings, the effect of 40% more hybridization time (56 hrs) was measured. Indeed, the lower dLRsd (0.16) indicated improved quality as expected (FIG. 6G), although detection algorithms did not yield additional information relative to the baseline data. It was concluded that increasing hybridization times improved data quality and could actually be beneficial when tissue and DNA quantity are limited but that the magnitude of such improvement was less than that imparted by fragment size matching (see FIGS. 6E-6F).

Finally, whether use of reference DNA of a more closely related tissue type and tissue fixation conditions might further improve results obtained from experimental samples was sought to be determined. Data obtained from competitive hybridization of an FFPE brain tumor sample (GBM2 DNA from a glioblastoma) and genomic DNA isolated from FFPE “normal” brain tissue showed little suggestion of further improvement in data quality (dLRsd=0.21).

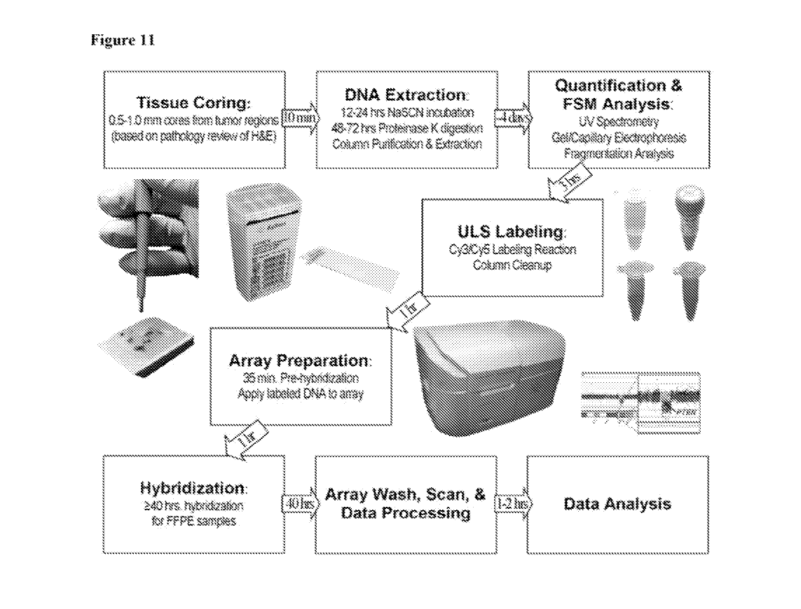

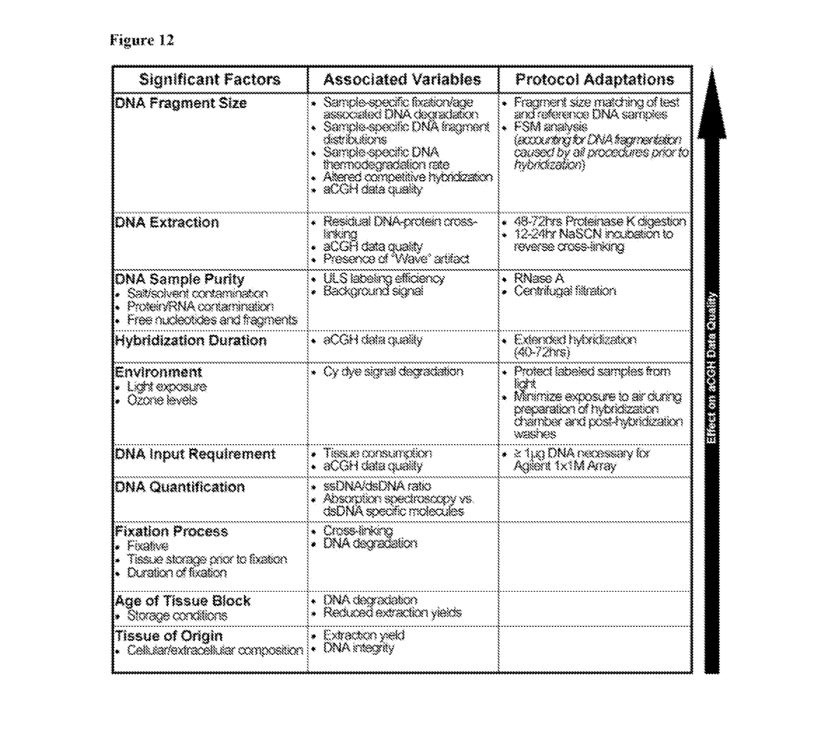

In summary, the use of FSM to match DNA fragment sizes (FIG. 11) unveiled a hierarchy of factors that affect the performance of aCGH (FIG. 12), and allows focused efforts to improve sample performance. Consequently, application of FSM expands the range of samples that can successfully be analyzed by aCGH.

Thus, these results identify some of the major sources of aCGH variability and provide new methods for improving the data generated from suboptimal DNA specimens. By using a single source of high quality reference genomic DNA and carefully controlling DNA fragmentation, it has been demonstrated herein that mismatched DNA fragment size distributions profoundly alter competitive hybridization under standard aCGH conditions more than previously suspected. These data are scientifically supported by previous biochemical studies which used short, fixed oligonucleotide probes to demonstrate that hybridization efficiency was inversely proportional to the length of the free (solution-side) end of the target strand in hybridizations. As a result, hybridization efficiency is significantly affected by DNA fragment length and the location of the hybridization along the length of the sequence (Peytavi et al. (2005) BioTech. 39:89-96). When interpreted in the context of competitive hybridization, the findings of Peytavi et al. suggest that the competition of genomic DNA fragments may be significantly influenced by size-dependent hybridization efficiencies. As a fundamental assumption underlying all CGH technology, equivalent hybridization properties of differentially labeled DNA fragments are necessary if concentration (i.e. copy number) is to be accurately reflected by signal intensity at equilibrium (Kallioniemi el al. (1992) Science 258:818-821). Therefore, without being bound by theory, it is believed that—by matching the DNA fragment sizes of both samples—it has been demonstrated herein that differences in hybridization efficiency have presumably been minimized and thereby promoted improved data quality.

Additionally, and without being bound by theory, it is believed that matching DNA fragment size within an optimal size range further increases the proportion of fragments that are viable hybridization targets and therefore increases the effective target concentration, driving the hybridization towards thermodynamic equilibrium. This effect can explain the high quality results generated by the FSM ULS method and why it also allows use of less sample DNA (FIG. 6F), similar to the manner in which extended hybridization improves data quality (FIG. 6G) by allowing the reaction to proceed closer to equilibrium.

Regardless of mechanism, the empirically demonstrated effect of matching fragment sizes in competitively hybridized DNA samples enabled application of the FSM ULS protocol and achievement of the robust aCGH data reported herein. The utility of FSM ULS also supports the substantial predictive power of prospective quality control assays (van Beers et al. (2006) Brit. J. Canc. 94:333-337; Johnson et al. (2006) Lab. Invest. 86:968-978; Buffart et al. (2007) Cell. Oncol. 29:351-359; and Alers et al. (1999) Genes, Chrom. Canc. 25:301-305). Since these latter assays based their sample selection criteria on indirect measures of DNA fragment size, each enabled a beneficial selection of samples with more appropriate and homogenous DNA fragment size distributions. Without being bound by theory, it is believed that the percentage of samples that failed to yield meaningful aCGH data in each study can be explained by unaccounted DNA fragmentation occurring during labeling, as well as by variable thermodegradation rates intrinsic to the sample (FIG. 3), and/or dissimilar reference DNA fragment distributions. DNA fragment size matching is also likely to have contributed to improved aCGH quality obtained in a recent study advocating application of DNase I fragmentation and enzymatic labeling (Hostetter et al. (2010) Nucl. Acids Res. 38:e9). Notably, this study is among several recent reports that have also attributed their improved aCGH performance with FFPE tissues to the labeling of increased amounts of sample DNA (as much as 5 μg for an Agilent 244 k array), a practice that has been cited as necessary to overcome the negative effects of the compromised template DNA (Al-Mulla (2011) Meth. Mol. Biol. 724:131-145; and Savage and Hostetter (2011) Meth. Mol. Biol. 700:185-198). While increasing the amount of DNA in the reaction may achieve similar results, the use of such large amounts of DNA is not generally practical for application to standard clinical samples where the amount of tissue available is limited, and current trends and future technologies will likely necessitate use of only nanogram amounts of DNA. While the methods described herein should allow the widest adoption by labs, it is believed that reductions in DNA requirements may be achieved with the FSM and other methods through use of low-sample volume capillary gel electrophoresis systems in the size modeling step. Additional reductions may come from the use of lower resolution arrays that are generally still of sufficient resolution to identify the majority of clinically relevant cancer aberrations.

Another likely source of improved results in the methods described is the preferred use of the chemically based ULS labeling method over enzymatic methods. Conceptually, ULS labeling is less affected by fixation-associated artifacts such as DNA cross-linking and DNA fragmentation. The ULS technology, which employs a platinum-based chemical reaction, adds Cy3 and Cy5 conjugates directly to the sample DNA at the N⁷ position of guanine bases, and also is independent of DNA strand length (van Gijlswijk et al. (2001) Exp. Rev. Mol. Diagnost. 1:81-91; and Heetebrij et al. (2003) Chembiochem 4:573-583). In contrast, enzymatic labeling further degrades the DNA during required denaturation steps (Gustafson et al. (1993) Gene 123:241-244), reduces the complexity of the original genomic template, and therefore may introduce bias in downstream copy number data (van Gijlswijk et al. (2001) Exp. Rev. Mol. Diagnost. 1:81-91). Yet despite the advantages of ULS labeling, use of this labeling approach is not as widely reported, particularly with intact DNA sources such as fresh tissues or blood (Hostetter et al. (2010) Nucl. Acids Res. 38:e9). Marked variation in performance of standard ULS labeled samples, consistent with the outcomes reported by Hostetter et al., was observed in the Examples described herein. As a result and without being bound by theory, it is believed that application of the FSM method was integral to the successful hybridization of relatively intact DNA because the appropriate fragmentation time required by a given sample was more variable than that of the FFPE derived samples (FIG. 4E). It is believed that the results described herein is one of the first large-scale studies to report the successful application of ULS labeling to high-resolution aCGH analysis of non-FFPE as well as FFPE DNA sources. The methodology may therefore allow a wider use of ULS technology, which offers distinct benefits of speed and simplified sample preparation across cancer and non-cancer applications (FIG. 11).

With regard to the fundamental suitability of FFPE samples for whole-genome analyses, the results with the FSM ULS protocol described herein indicate that FFPE DNA is not damaged in any way that irreversibly affects aCGH performance, but methods to account for the decreased DNA fragment size encountered must be more routinely implemented. The correlation between FFPE block age and increased fragmentation is consistent with the lower success rates previously reported with older samples when fragment size was not carefully controlled, but the results described herein indicate that recommendations that samples older than 10 years of age should be excluded from research or clinical analysis need to be reevaluated. Future analysis of samples beyond 15 years of age may aid in determining whether an upper age limit might exist for FFPE specimens analyzed by aCGH using FSM or other methods. Notably, while the Agilent stock 1 M feature array offers extremely high resolution and a genome wide median probe spacing of 2.1 kb, the enhanced resolution confers greater sensitivity to both true copy number alterations as well as “noise” when compared with lower resolution arrays such as the Agilent 244 k array (Al-Mulla (2011) Meth. Mol. Biol. 724:131-145; and Przybytkowski et al. (2011) BMC Med. Genom. 4:16). The choice of the Agilent 1 M array for quality comparisons therefore represents a significantly stringent standard for any aCGH method and the fact that uniform dLRsd below 0.3 was achieved over large and diverse samples sets from multiple international institutions using a wide range of fixation conditions again indicates that the array type and other variables are potentially minor variables in array performance relative to sample preparation and hybridization conditions.

In developing the FSM methodology, the protocol was optimized to use commonplace and affordable laboratory equipment and to not require complex procedures. Although the method uses heat fragmentation, other methods that allow greater control over matched DNA fragment distributions could also be used successfully. Sample methods that utilize shearing (e.g., Covaris technology, such as adaptive focused acoustics-based shearing), restriction enzyme, or size selection approaches would be useful to compare to the results from heat fragmentation reported here. Application of FSM methodology to standardization of genomic DNA ensures that powerful FFPE-compatible diagnostic laboratory tools can more easily be implemented into routine clinical use. The FSM approach of modeling nucleic acid fragmentation to predict downstream fragment sizes may also have utility for other hybridization-based reactions, such as Affymetrix SNP arrays and Nanostring arrays, or hybrid capture methods commonly used in next generation sequencing, exome sequencing, and the like.

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

TABLE 1
Sample Summary

| | | | | Block | | | |
|---|---|---|---|---|---|---|---|
| Study ID | | | | Age | Sample | | Array |
| (Craig_FSM_XXX) | Source | Diagnosis | dLRsd | (yrs) | Type | Method | Version |
| Craig_FSM_001 | JHMI | LGG | 0.18 | 3 | FFPE | FSM | Agilent 1M |
| Craig_FSM_002 | JHMI | LGG | 0.18 | 7 | FFPE | FSM | Agilent 1M |
| Craig_FSM_003 | JHMI | LGG | 0.23 | 10 | FFPE | FSM | Agilent 1M |
| Craig_FSM_004 | JHMI | LGG | 0.23 | 11 | FFPE | FSM | Agilent 1M |
| Craig_FSM_005 | BWH | Metastasis, | 0.18 | 0.5 | FFPE | FSM | Agilent 1M |
| | | Breast | | | | | |
| Craig_FSM_006 | BWH | Metastasis, | 0.17 | 0.5 | FFPE | FSM | Agilent 1M |
| | | Breast | | | | | |
| Craig_FSM_007 | BWH | Metastasis, | 0.17 | 0.5 | FFPE | FSM | Agilent 1M |
| | | Breast | | | | | |
| Craig_FSM_008 | CHB | LGG | 0.19 | 0.5 | FFPE | FSM | Agilent 1M |
| Craig_FSM_009 | CHB | LGG | 0.18 | 0.5 | FFPE | FSM | Agilent 1M |
| Craig_FSM_010 | CHB | A4 | 0.23 | 0.5 | FFPE | FSM | Agilent 1M |
| Craig_FSM_011 | CHB | LGG | 0.17 | 1 | FFPE | FSM | Agilent 1M |
| Craig_FSM_012 | CHB | LGG | 0.18 | 1 | FFPE | FSM | Agilent 1M |
| Craig_FSM_013 | CHB | LGG | 0.23 | 4 | FFPE | FSM | Agilent 1M |
| Craig_FSM_014 | CHB | LGG | 0.20 | 5 | FFPE | FSM | Agilent 1M |
| Craig_FSM_015 | CHB | LGG | 0.17 | 7 | FFPE | FSM | Agilent 1M |
| Craig_FSM_016 | CHB | LGG | 0.19 | 7 | FFPE | FSM | Agilent 1M |
| Craig_FSM_017 | CHB | LGG | 0.21 | 7 | FFPE | FSM | Agilent 1M |
| Craig_FSM_018 | CHB | LGG | 0.18 | 8 | FFPE | FSM | Agilent 1M |
| Craig_FSM_019 | CHB | LGG | 0.22 | 8 | FFPE | FSM | Agilent 1M |
| Craig_FSM_020 | CHB | LGG | 0.23 | 9 | FFPE | FSM | Agilent 1M |
| Craig_FSM_021 | CHB | LGG | 0.19 | 11 | FFPE | FSM | Agilent 1M |
| Craig_FSM_022 | CHB | LGG | 0.21 | 13 | FFPE | FSM | Agilent 1M |
| Craig_FSM_023 | CHB | LGG | 0.19 | 8 | FFPE | FSM | Agilent 1M |
| Craig_FSM_024 | CNMC | LGG | 0.29 | 2 | FFPE | FSM | Agilent 1M |
| Craig_FSM_025 | CNMC | LGG | 0.22 | 2 | FFPE | FSM | Agilent 1M |
| Craig_FSM_026 | CNMC | LGG | 0.23 | 3 | FFPE | FSM | Agilent 1M |
| Craig_FSM_027 | CNMC | LGG | 0.20 | 4 | FFPE | FSM | Agilent 1M |
| Craig_FSM_028 | CNMC | LGG | 0.22 | 5 | FFPE | FSM | Agilent 1M |
| Craig_FSM_029 | CNMC | LGG | 0.20 | 7 | FFPE | FSM | Agilent 1M |
| Craig_FSM_030 | CNMC | LGG | 0.20 | 8 | FFPE | FSM | Agilent 1M |
| Craig_FSM_031 | CMCD | LGG | 0.21 | 0 | FFPE | FSM | Agilent 1M |
| Craig_FSM_032 | CMCD | LGG | 0.15 | 1 | FFPE | FSM | Agilent 1M |
| Craig_FSM_033 | CMCD | LGG | 0.17 | 2 | FFPE | FSM | Agilent 1M |
| Craig_FSM_034 | CMCD | LGG | 0.15 | 2 | FFPE | FSM | Agilent 1M |
| Craig_FSM_035 | CMCD | LGG | 0.27 | 2 | FFPE | FSM | Agilent 1M |
| Craig_FSM_036 | CMCD | LGG | 0.18 | 3 | FFPE | FSM | Agilent 1M |
| Craig_FSM_037 | CMCD | LGG | 0.16 | 4 | FFPE | FSM | Agilent 1M |
| Craig_FSM_038 | CMCD | LGG | 0.20 | 5 | FFPE | FSM | Agilent 1M |
| Craig_FSM_039 | CMCD | LGG | 0.17 | 5 | FFPE | FSM | Agilent 1M |
| Craig_FSM_040 | CMCD | LGG | 0.21 | 5 | FFPE | FSM | Agilent 1M |
| Craig_FSM_041 | CMCD | LGG | 0.17 | 6 | FFPE | FSM | Agilent 1M |
| Craig_FSM_042 | CMCD | LGG | 0.17 | 7 | FFPE | FSM | Agilent 1M |
| Craig_FSM_043 | CMCD | LGG | 0.16 | 8 | FFPE | FSM | Agilent 1M |
| Craig_FSM_044 | CMCD | LGG | 0.20 | 8 | FFPE | FSM | Agilent 1M |
| Craig_FSM_045 | CHB | LGG | 0.24 | 10 | FFPE | FSM | Agilent 1M |
| Craig_FSM_046 | CHB | LGG | 0.27 | 11 | FFPE | FSM | Agilent 1M |
| Craig_FSM_047 | CHB | LGG | 0.26 | 11 | FFPE | FSM | Agilent 1M |
| Craig_FSM_048 | CHB | LGG | 0.19 | 11 | FFPE | FSM | Agilent 1M |
| Craig_FSM_049 | IST | LGG | 0.22 | 12 | FFPE | FSM | Agilent 1M |
| Craig_FSM_050 | IST | LGG | 0.22 | 13 | FFPE | FSM | Agilent 1M |
| Craig_FSM_051 | IST | LGG | 0.21 | 13 | FFPE | FSM | Agilent 1M |
| Craig_FSM_052 | BWH | O3 | 0.23 | 5 | FFPE | FSM | Agilent 1M |
| Craig_FSM_053 | BWH | Normal/Other | 0.20 | 2 | FFPE | FSM | Agilent 1M |
| Craig_FSM_054 | BWH | Normal/Other | 0.25 | 3 | FFPE | FSM | Agilent 1M |
| Craig_FSM_055 | BWH | Normal/Other | 0.23 | 3 | FFPE | FSM | Agilent 1M |
| Craig_FSM_056 | BWH | Normal/Other | 0.26 | 4 | FFPE | FSM | Agilent 1M |
| Craig_FSM_057 | BWH | A4 | 0.24 | 6 | FFPE | FSM | Agilent 1M |
| Craig_FSM_058 | BWH | Normal/Other | 0.20 | 6 | FFPE | FSM | Agilent 1M |
| Craig_FSM_059 | BWH | A4 | 0.24 | 2 | FFPE | FSM | Agilent 1M |
| Craig_FSM_060 | BWH | A4 | 0.20 | 1 | FFPE | FSM | Agilent 1M |
| Craig_FSM_061 | BWH | A4 | 0.20 | 1 | FFPE | FSM | Agilent 1M |
| Craig_FSM_062 | BWH | A4 | 0.17 | 2 | FFPE | FSM | Agilent 1M |
| Craig_FSM_063 | BWH | A4 | 0.16 | 2 | FFPE | FSM | Agilent 1M |
| Craig_FSM_064 | BWH | A4 | 0.15 | 2 | FFPE | FSM | Agilent 1M |
| Craig_FSM_065 | BWH | A4 | 0.18 | 2 | FFPE | FSM | Agilent 1M |
| Craig_FSM_066 | BWH | A4 | 0.16 | 2 | FFPE | FSM | Agilent 1M |
| Craig_FSM_067 | BWH | A4 | 0.20 | 2 | FFPE | FSM | Agilent 1M |
| Craig_FSM_068 | BWH | A4 | 0.24 | 2 | FFPE | FSM | Agilent 1M |
| Craig_FSM_069 | BWH | A4 | 0.15 | 2 | FFPE | FSM | Agilent 1M |
| Craig_FSM_070 | BWH | A4 | 0.19 | 2 | FFPE | FSM | Agilent 1M |
| Craig_FSM_071 | BWH | A4 | 0.17 | 3 | FFPE | FSM | Agilent 1M |
| Craig_FSM_072 | BWH | A4 | 0.18 | 3 | FFPE | FSM | Agilent 1M |
| Craig_FSM_073 | BWH | A4 | 0.22 | 3 | FFPE | FSM | Agilent 1M |
| Craig_FSM_074 | BWH | A4 | 0.19 | 3 | FFPE | FSM | Agilent 1M |
| Craig_FSM_075 | BWH | A4 | 0.18 | 3 | FFPE | FSM | Agilent 1M |
| Craig_FSM_076 | BWH | A4 | 0.18 | 3 | FFPE | FSM | Agilent 1M |
| Craig_FSM_077 | BWH | A4 | 0.21 | 2 | FFPE | FSM | Agilent 1M |
| Craig_FSM_078 | BWH | A4 | 0.22 | 1 | FFPE | FSM | Agilent 1M |
| Craig_FSM_079 | BWH | A4 | 0.21 | 1 | FFPE | FSM | Agilent 1M |
| Craig_FSM_080 | BWH | A4 | 0.18 | 1 | FFPE | FSM | Agilent 1M |
| Craig_FSM_081 | CHB | LGG | 0.78 | 7 | FFPE | STANDARD | Agilent 1M |
| Craig_FSM_082 | CHB | LGG | 0.31 | 8 | FFPE | STANDARD | Agilent 1M |
| Craig_FSM_083 | CHB | LGG | 0.30 | 8 | FFPE | STANDARD | Agilent 1M |
| Craig_FSM_084 | CHB | LGG | 0.32 | 8 | FFPE | STANDARD | Agilent 1M |
| Craig_FSM_085 | CHB | LGG | 0.48 | 8 | FFPE | STANDARD | Agilent 1M |
| Craig_FSM_086 | CHB | LGG | 0.28 | 10 | FFPE | STANDARD | Agilent 1M |
| Craig_FSM_087 | CHB | LGG | 0.34 | 10 | FFPE | STANDARD | Agilent 1M |
| Craig_FSM_088 | CHB | LGG | 0.40 | 10 | FFPE | STANDARD | Agilent 1M |
| Craig_FSM_089 | CHB | LGG | 0.46 | 11 | FFPE | STANDARD | Agilent 1M |
| Craig_FSM_090 | CHB | LGG | 0.65 | 12 | FFPE | STANDARD | Agilent 1M |
| Craig_FSM_091 | CHB | LGG | 0.37 | 14 | FFPE | STANDARD | Agilent 1M |
| Craig_FSM_092 | CHB | LGG | 0.27 | 14 | FFPE | STANDARD | Agilent 1M |
| Craig_FSM_093 | CHB | LGG | 0.35 | 14 | FFPE | STANDARD | Agilent 1M |
| Craig_FSM_094 | CHB | LGG | 0.30 | 15 | FFPE | STANDARD | Agilent 1M |
| Craig_FSM_095 | CHB | LGG | 0.63 | 15 | FFPE | STANDARD | Agilent 1M |
| Craig_FSM_096 | CHB | LGG | 0.35 | 15 | FFPE | STANDARD | Agilent 1M |
| Craig_FSM_097 | BWH | Normal/Other | 0.24 | 4 | FFPE | STANDARD | Agilent 1M |
| Craig_FSM_098 | BWH | A4 | 0.27 | 2 | FFPE | STANDARD | Agilent 1M |
| Craig_FSM_099 | BWH | Normal/Other | 0.22 | 4 | FFPE | STANDARD | Agilent 1M |
| Craig_FSM_100 | BWH | Normal/Other | 0.20 | 4 | FFPE | STANDARD | Agilent 1M |
| Craig_FSM_101 | CHB | LGG | 0.32 | 3 | FFPE | STANDARD | Agilent 1M |
| Craig_FSM_102 | CHB | LGG | 0.37 | 9 | FFPE | STANDARD | Agilent 1M |
| Craig_FSM_103 | BWH | Normal/Other | 0.43 | 4 | FFPE | STANDARD | Agilent 1M |
| Craig_FSM_104 | BWH | A4 | 0.46 | 1 | FFPE | STANDARD | Agilent 1M |
| Craig_FSM_105 | BWH | A4 | 0.37 | 1 | FFPE | STANDARD | Agilent 1M |
| Craig_FSM_106 | BWH | LGG | 0.31 | 1 | FFPE | STANDARD | Agilent 1M |
| Craig_FSM_107 | BWH | O3 | 0.29 | 1 | FFPE | STANDARD | Agilent 1M |
| Craig_FSM_108 | BWH | A4 | 0.26 | 2 | FFPE | STANDARD | Agilent 1M |
| Craig_FSM_109 | BWH | O3 | 0.24 | 2 | FFPE | STANDARD | Agilent 1M |
| Craig_FSM_110 | BWH | A4 | 0.21 | 2 | FFPE | STANDARD | Agilent 1M |
| Craig_FSM_111 | BWH | A4 | 0.32 | 2 | FFPE | STANDARD | Agilent 1M |
| Craig_FSM_112 | BWH | A4 | 0.40 | 2 | FFPE | STANDARD | Agilent 1M |
| Craig_FSM_113 | BWH | LGG | 0.33 | 2 | FFPE | STANDARD | Agilent 1M |
| Craig_FSM_114 | BWH | A4 | 0.34 | 2 | FFPE | STANDARD | Agilent 1M |
| Craig_FSM_115 | BWH | LGG | 0.32 | 2 | FFPE | STANDARD | Agilent 1M |
| Craig_FSM_116 | BWH | A4 | 0.31 | 2 | FFPE | STANDARD | Agilent 1M |
| Craig_FSM_117 | BWH | A4 | 0.35 | 2 | FFPE | STANDARD | Agilent 1M |
| Craig_FSM_118 | BWH | A4 | 0.46 | 2 | FFPE | STANDARD | Agilent 1M |
| Craig_FSM_119 | BWH | A4 | 0.34 | 3 | FFPE | STANDARD | Agilent 1M |
| Craig_FSM_120 | CHB | LGG | 0.26 | 3 | FFPE | STANDARD | Agilent 1M |
| Craig_FSM_121 | BWH | Normal/Other | 0.47 | 4 | FFPE | STANDARD | Agilent 1M |
| Craig_FSM_122 | BWH | O3 | 0.57 | 15 | FFPE | STANDARD | Agilent 1M |
| Craig_FSM_123 | DF/HCC | A4 | 0.20 | N/A | CELLS | FSM | Agilent 1M |
| Craig_FSM_124 | DF/HCC | A4 | 0.18 | N/A | CELLS | FSM | Agilent 1M |
| Craig_FSM_125 | DF/HCC | A4 | 0.18 | N/A | CELLS | FSM | Agilent 1M |
| Craig_FSM_126 | DF/HCC | A4 | 0.16 | N/A | CELLS | FSM | Agilent 1M |
| Craig_FSM_127 | DF/HCC | A4 | 0.18 | N/A | CELLS | FSM | Agilent 1M |
| Craig_FSM_128 | DF/HCC | A4 | 0.22 | N/A | CELLS | FSM | Agilent 1M |
| Craig_FSM_129 | DF/HCC | A4 | 0.15 | N/A | CELLS | FSM | Agilent 1M |
| Craig_FSM_130 | DF/HCC | A4 | 0.21 | N/A | CELLS | FSM | Agilent 1M |
| Craig_FSM_131 | DF/HCC | A4 | 0.22 | N/A | CELLS | FSM | Agilent 1M |
| Craig_FSM_132 | DF/HCC | A4 | 0.19 | N/A | CELLS | FSM | Agilent 1M |
| Craig_FSM_133 | DF/HCC | A4 | 0.18 | N/A | CELLS | FSM | Agilent 1M |
| Craig_FSM_134 | DF/HCC | A4 | 0.18 | N/A | CELLS | FSM | Agilent 1M |
| Craig_FSM_135 | DF/HCC | A4 | 0.18 | N/A | CELLS | FSM | Agilent 1M |
| Craig_FSM_136 | DF/HCC | LGG | 0.28 | N/A | CELLS | FSM | Agilent 1M |
| Craig_FSM_137 | DF/HCC | A4 | 0.18 | N/A | CELLS | FSM | Agilent 1M |
| Craig_FSM_138 | DF/HCC | A4 | 0.20 | N/A | CELLS | FSM | Agilent 1M |
| Craig_FSM_139 | DF/HCC | A4 | 0.22 | N/A | CELLS | FSM | Agilent 1M |
| Craig_FSM_140 | DF/HCC | O3 | 0.24 | N/A | CELLS | FSM | Agilent 1M |
| Craig_FSM_141 | DF/HCC | O3 | 0.15 | N/A | CELLS | FSM | Agilent 1M |
| Craig_FSM_142 | DF/HCC | A3 | 0.23 | N/A | CELLS | FSM | Agilent 1M |
| Craig_FSM_143 | DF/HCC | O3 | 0.22 | N/A | CELLS | FSM | Agilent 1M |
| Craig_FSM_144 | DF/HCC | A4 | 0.22 | N/A | CELLS | FSM | Agilent 1M |
| Craig_FSM_145 | DF/HCC | A4 | 0.18 | N/A | CELLS | FSM | Agilent 1M |
| Craig_FSM_146 | DF/HCC | A4 | 0.17 | N/A | CELLS | FSM | Agilent 1M |
| Craig_FSM_147 | DF/HCC | LGG | 0.18 | N/A | CELLS | FSM | Agilent 1M |
| Craig_FSM_148 | DF/HCC | LGG | 0.17 | N/A | CELLS | FSM | Agilent 1M |
| Craig_FSM_149 | DF/HCC | A4 | 0.15 | N/A | CELLS | FSM | Agilent 1M |
| Craig_FSM_150 | DF/HCC | A4 | 0.15 | N/A | CELLS | FSM | Agilent 1M |
| Craig_FSM_151 | DF/HCC | A4 | 0.22 | N/A | CELLS | FSM | Agilent 1M |
| Craig_FSM_152 | DF/HCC | A4 | 0.18 | N/A | CELLS | FSM | Agilent 1M |
| Craig_FSM_153 | CHB | LGG | 0.15 | 5 | FROZEN | FSM | Agilent 1M |
| Craig_FSM_154 | CHB | LGG | 0.15 | 5 | FROZEN | FSM | Agilent 1M |
| Craig_FSM_155 | CHB | LGG | 0.13 | 5 | FROZEN | FSM | Agilent 1M |
| Craig_FSM_156 | CHB | LGG | 0.13 | 4 | FROZEN | FSM | Agilent 1M |
| Craig_FSM_157 | CHB | LGG | 0.12 | 4 | FROZEN | FSM | Agilent 1M |
| Craig_FSM_158 | CHB | LGG | 0.14 | 4 | FROZEN | FSM | Agilent 1M |
| Craig_FSM_159 | CHB | LGG | 0.14 | 3 | FROZEN | FSM | Agilent 1M |
| Craig_FSM_160 | CHB | LGG | 0.14 | 2 | FROZEN | FSM | Agilent 1M |
| Craig_FSM_161 | UCSF | A4 | 0.15 | N/A | CELLS | FSM | Agilent 1M |
| Craig_FSM_162 | UCSF | A4 | 0.17 | N/A | CELLS | FSM | Agilent 1M |
| Craig_FSM_163 | UCSF | A4 | 0.15 | N/A | CELLS | FSM | Agilent 1M |
| Craig_FSM_164 | UCSF | A4 | 0.21 | N/A | CELLS | FSM | Agilent 1M |
| Craig_FSM_165 | UCSF | A4 | 0.15 | N/A | CELLS | FSM | Agilent 1M |
| Craig_FSM_166 | UCSF | A4 | 0.16 | N/A | CELLS | FSM | Agilent 1M |
| Craig_FSM_167 | UCSF | A4 | 0.25 | N/A | CELLS | FSM | Agilent 1M |
| Craig_FSM_168 | DF/HCC | A4 | 0.17 | N/A | CELLS | FSM | Agilent 1M |
| Craig_FSM_169 | DF/HCC | A4 | 0.15 | N/A | CELLS | FSM | Agilent 1M |
| Craig_FSM_170 | DF/HCC | A4 | 0.16 | N/A | CELLS | FSM | Agilent 1M |
| Craig_FSM_171 | DF/HCC | A4 | 0.17 | N/A | CELLS | FSM | Agilent 1M |
| Craig_FSM_172 | DF/HCC | A4 | 0.19 | N/A | CELLS | STANDARD | Agilent 1M |
| Craig_FSM_173 | DF/HCC | A4 | 0.21 | N/A | CELLS | STANDARD | Agilent 1M |
| Craig_FSM_174 | DF/HCC | A4 | 0.21 | N/A | CELLS | STANDARD | Agilent 1M |
| Craig_FSM_175 | DF/HCC | A4 | 0.22 | N/A | CELLS | STANDARD | Agilent 1M |
| Craig_FSM_176 | DF/HCC | O3 | 0.24 | N/A | CELLS | STANDARD | Agilent 1M |
| Craig_FSM_177 | DF/HCC | A4 | 0.24 | N/A | CELLS | STANDARD | Agilent 1M |
| Craig_FSM_178 | DF/HCC | A4 | 0.24 | N/A | CELLS | STANDARD | Agilent 1M |
| Craig_FSM_179 | DF/HCC | A4 | 0.25 | N/A | CELLS | STANDARD | Agilent 1M |
| Craig_FSM_180 | DF/HCC | A4 | 0.25 | N/A | CELLS | STANDARD | Agilent 1M |
| Craig_FSM_181 | DF/HCC | A4 | 0.26 | N/A | CELLS | STANDARD | Agilent 1M |
| Craig_FSM_182 | DF/HCC | A4 | 0.28 | N/A | CELLS | STANDARD | Agilent 1M |
| Craig_FSM_183 | DF/HCC | A4 | 0.28 | N/A | CELLS | STANDARD | Agilent 1M |
| Craig_FSM_184 | DF/HCC | A4 | 0.29 | N/A | CELLS | STANDARD | Agilent 1M |
| Craig_FSM_185 | DF/HCC | LGG | 0.29 | N/A | CELLS | STANDARD | Agilent 1M |
| Craig_FSM_186 | DF/HCC | A4 | 0.31 | N/A | CELLS | STANDARD | Agilent 1M |
| Craig_FSM_187 | DF/HCC | A4 | 0.31 | N/A | CELLS | STANDARD | Agilent 1M |
| Craig_FSM_188 | DF/HCC | A4 | 0.32 | N/A | CELLS | STANDARD | Agilent 1M |
| Craig_FSM_189 | DF/HCC | A4 | 0.32 | N/A | CELLS | STANDARD | Agilent 1M |
| Craig_FSM_190 | DF/HCC | A4 | 0.32 | N/A | CELLS | STANDARD | Agilent 1M |
| Craig_FSM_191 | DF/HCC | A4 | 0.33 | N/A | CELLS | STANDARD | Agilent 1M |
| Craig_FSM_192 | DF/HCC | A4 | 0.34 | N/A | CELLS | STANDARD | Agilent 1M |
| Craig_FSM_193 | DF/HCC | A4 | 0.35 | N/A | CELLS | STANDARD | Agilent 1M |
| Craig_FSM_194 | DF/HCC | O3 | 0.39 | N/A | CELLS | STANDARD | Agilent 1M |
| Craig_FSM_195 | DF/HCC | A4 | 0.40 | N/A | CELLS | STANDARD | Agilent 1M |
| Craig_FSM_196 | DF/HCC | A4 | 0.43 | N/A | CELLS | STANDARD | Agilent 1M |
| Craig_FSM_197 | DF/HCC | A4 | 0.58 | N/A | CELLS | STANDARD | Agilent 1M |
| Craig_FSM_198 | DF/HCC | A4 | 0.63 | N/A | CELLS | STANDARD | Agilent 1M |
| Craig_FSM_199 | DF/HCC | A4 | 0.68 | N/A | CELLS | STANDARD | Agilent 1M |
| Craig_FSM_200 | DF/HCC | A4 | 0.81 | N/A | CELLS | STANDARD | Agilent 1M |

Key:
BWH [Brigham and Women's Hospital (Boston, MA)]
CHB [Children's Hospital Boston (Boston, MA)]
CMCD [Children's Medical Center in Dallas (Dallas, TX)]
CNMC [Children's National Medical Center (Washington, D.C.)]
DF/HCC [Dana Farber/Harvard Cancer Center (Boston, MA)]
IST [Marmara University Medical Center (Istanbul, Turkey)]
JHMI [John Hopkins Medical Institutes (Baltimore, MD)]
UCSF [University of California San Francisco (San Francisco, CA)]
A3 [astrocytoma, Grade III]
A4 [astrocytoma, Grade IV]
O3 [anaplastic oligodendroglioma]
LGG [low grade glioma]
Metastasis, Breast [metastatic breast cancer in brain]
Normal/Other [normal, non-diagnostic brian tissue or epilepsy biopsy tissue]
FFPE [formalin-fixed paraffin-embedded brain tissue]
FROZEN [tumor tissue frozen at time of resection]
CELLS [fresh-frozen cell cultures]
FSM [array processed according to FSM ULS protocol]
STANDARD [array processed acording to standard ULS protocol]

TABLE 2
Additional Quality Control (QC) Metrics

| | | | Signal To | Signal To | Signal | Signal | BG | BG | Reproduc- | Reproduc- |
|---|---|---|---|---|---|---|---|---|---|---|
| | | | Noise | Noise | Intensity | Intensity | Noise | Noise | ibility | ibility |
| Study ID | Description | dt.Rsd | Green | Red | Green | Red | Gree | Red | Green | Red |
| Fig_1B & 2I | 225/225 (bp) self-hyb | 0.26 | 13.38 | 18.82 | 41.43 | 95.83 | 3.10 | 5.00 | 12.14 | 12.88 |
| Fig_1D | 225/225 (bp) self-hyb | 0.32 | 17.86 | 40.85 | 59.26 | 315.13 | 3.32 | 7.71 | 12.63 | 11.03 |
| Fig_1F | 140/225 (bp) self-hyb | 0.42 | 50.72 | 9.79 | 213.16 | 47.49 | 4.20 | 4.85 | 10.56 | 14.09 |
| Fig_1H | 140/225 (bp) self-hyb | 0.74 | 2.13 | 3.70 | 8.19 | 17.18 | 3.23 | 4.65 | 19.38 | 15.72 |
| Fig_2I | 685/685 (bp) self-hyb | 0.16 | 58.80 | 38.83 | 230.26 | 173.08 | 3.92 | 4.46 | 10.88 | 12.29 |
| Fig_2I | 685/685 (bp) self-hyb | 0.16 | 51.29 | 34.85 | 260.73 | 169.78 | 3.08 | 4.87 | 11.19 | 12.90 |
| Fig_2I | 625/625 (bp) self-hyb | 0.16 | 50.60 | 34.65 | 170.09 | 160.05 | 3.36 | 4.33 | 9.16 | 10.83 |
| Fig_2I | 625/625 (bp) self-hyb | 0.16 | 52.58 | 33.42 | 202.86 | 142.41 | 3.86 | 4.20 | 12.36 | 12.80 |
| Fig_2H & 2I | 525/525 (bp) self-hyb | 0.15 | 41.20 | 33.29 | 218.89 | 149.04 | 4.84 | 4.48 | 12.23 | 11.93 |
| Fig_2I | 525/525 (bp) self-hyb | 0.15 | 57.10 | 33.02 | 191.76 | 133.00 | 3.35 | 4.05 | 10.73 | 13.72 |
| Fig_2I | 525/525 (bp) self-hyb | 0.16 | 42.48 | 29.04 | 190.24 | 130.25 | 4.48 | 4.49 | 11.26 | 12.50 |
| Fig_2F & 2I | 400/400 (bp) self-hyb | 0.16 | 45.36 | 29.47 | 199.83 | 123.92 | 4.41 | 4.20 | 10.34 | 13.23 |
| Fig_2I | 400/400 (bp) self-hyb | 0.16 | 47.37 | 30.86 | 172.41 | 122.55 | 3.64 | 3.97 | 11.05 | 14.11 |
| Fig_2I | 400/400 (bp) self-hyb | 0.16 | 40.24 | 25.74 | 133.82 | 121.19 | 3.33 | 4.71 | 10.21 | 12.09 |
| Fig_2D & 2I | 315/315 (bp) self-hyb | 0.18 | 32.42 | 23.80 | 115.75 | 116.25 | 3.57 | 4.88 | 12.10 | 13.00 |
| Fig_2I | 315/315 (bp) self-hyb | 0.19 | 22.42 | 17.79 | 102.10 | 80.38 | 3.72 | 5.02 | 13.07 | 14.97 |
| Fig_2I | 250/250 (bp) self-hyb | 0.22 | 31.01 | 22.14 | 90.20 | 94.6 | 2.85 | 4.27 | 10.04 | 12.05 |
| Fig_2B & 2I | 250/250 (bp) self-hyb | 0.24 | 22.20 | 14.81 | 74.95 | 69.43 | 3.38 | 4.69 | 12.19 | 14.95 |
| Fig_2I | 225/225 (bp) self-hyb | 0.26 | 28.72 | 30.71 | 82.01 | 131.72 | 2.80 | 4.29 | 12.01 | 12.66 |
| Fig_6A | GBM1/FSM | 0.16 | 44.35 | 36.46 | 179.01 | 185.32 | 4.02 | 4.02 | 14.70 | 14.89 |
| Fig_6B | GBM1/Standard | 0.44 | 40.94 | 14.83 | 136.33 | 66.67 | 3.31 | 4.50 | 56.13 | 54.50 |
| Fig_6C | GBM1/FSM/15 hr ProK | 0.23 | 41.44 | 20.63 | 196.50 | 83.13 | 4.74 | 4.03 | 13.23 | 15.79 |
| Fig_6D | GBM2/FSM/2.0 ug | 0.20 | 37.16 | 31.93 | 207.41 | 145.92 | 3.58 | 4.57 | 12.37 | 13.62 |
| Fig_6E | GBM2/FSM/3.5 ug | 0.23 | 33.98 | 28.32 | 168.89 | 113.41 | 4.97 | 4.03 | 16.29 | 16.41 |
| Fig_6F | GBM2/FSM/3.0 ug | 0.27 | 25.84 | 20.77 | 104.34 | 74.72 | 4.04 | 3.60 | 16.22 | 17.84 |
| Fig_6G | GBM2/FSM/2.0 ug/ext hyb | 0.16 | 29.50 | 31.95 | 162.56 | 167.00 | 4.12 | 5.25 | 14.59 | 14.40 |
| Fig_6H | GBM2/FSM/2.0 ug/TTPE ref | 0.21 | 27.18 | 32.09 | 115.10 | 144.12 | 4.23 | 4.41 | 0.15 | 0.16 |
| Craig_FSM_001 | FFPE/FSM | 0.18 | 38.45 | 27.34 | 169.00 | 167.51 | 4.40 | 4.13 | 14.83 | 14.98 |
| Craig_FSM_002 | FFPE/FSM | 0.18 | 39.11 | 27.37 | 174.94 | 166.66 | 4.47 | 6.00 | 13.06 | 13.67 |
| Craig_FSM_003 | FFPE/FSM | 0.23 | 35.84 | 23.62 | 152.59 | 137.08 | 4.26 | 5.80 | 12.16 | 13.01 |
| Craig_FSM_004 | FFPE/FSM | 0.23 | 38.60 | 22.55 | 170.48 | 142.14 | 4.42 | 6.30 | 15.49 | 15.92 |
| Craig_FSM_005 | FFPE/FSM | 0.18 | 32.63 | 26.11 | 279.15 | 216.77 | 7.42 | 6.00 | 15.00 | 16.13 |
| Craig_FSM_006 | FFPE/FSM | 0.17 | 43.06 | 37.65 | 264.00 | 247.51 | 6.13 | 6.57 | 15.43 | 15.18 |
| Craig_FSM_007 | FFPE/FSM | 0.17 | 46.78 | 36.17 | 220.91 | 189.28 | 4.72 | 5.23 | 13.52 | 14.88 |
| Craig_FSM_008 | FFPE/FSM | 0.19 | 51.51 | 30.73 | 222.19 | 123.56 | 4.31 | 4.02 | 13.11 | 17.03 |
| Craig_FSM_009 | FFPE/FSM | 0.18 | 53.74 | 31.48 | 234.62 | 125.05 | 4.37 | 3.97 | 14.21 | 23.45 |
| Craig_FSM_010 | FFPE/FSM | 0.23 | 52.18 | 25.94 | 214.84 | 95.76 | 4.12 | 3.69 | 13.39 | 18.49 |
| Craig_FSM_011 | FFPE/FSM | 0.37 | 42.47 | 28.31 | 185.06 | 155.82 | 4.36 | 5.50 | 16.16 | 17.04 |
| Craig_FSM_012 | FFPE/FSM | 0.38 | 56.21 | 32.31 | 237.16 | 139.88 | 4.22 | 4.36 | 14.87 | 18.72 |
| Craig_FSM_013 | FFPE/FSM | 0.23 | 51.69 | 20.56 | 233.85 | 83.36 | 4.52 | 4.05 | 13.91 | 18.92 |
| Craig_FSM_014 | FFPE/FSM | 0.20 | 39.34 | 21.44 | 148.42 | 114.93 | 3.77 | 5.36 | 16.69 | 18.03 |
| Craig_FSM_015 | FFPE/FSM | 0.17 | 41.92 | 29.78 | 181.10 | 169.74 | 4.32 | 5.40 | 15.42 | 16.04 |
| Craig_FSM_016 | FFPE/FSM | 0.19 | 40.99 | 26.19 | 190.78 | 155.13 | 4.65 | 5.92 | 16.56 | 17.89 |
| Craig_FSM_017 | FFPE/FSM | 0.21 | 41.99 | 22.44 | 204.21 | 104.40 | 4.86 | 4.05 | 12.91 | 16.12 |
| Craig_FSM_018 | FFPE/FSM | 0.18 | 41.14 | 25.92 | 159.55 | 134.54 | 3.88 | 5.19 | 13.90 | 15.66 |
| Craig_FSM_019 | FFPE/FSM | 0.22 | 42.50 | 23.61 | 213.20 | 108.19 | 5.02 | 4.58 | 14.98 | 16.12 |
| Craig_FSM_020 | FFPE/FSM | 0.23 | 40.88 | 18.01 | 182.87 | 106.35 | 4.47 | 5.90 | 18.80 | 21.76 |
| Craig_FSM_021 | FFPE/FSM | 0.19 | 38.81 | 25.05 | 178.14 | 154.36 | 4.59 | 6.16 | 12.87 | 14.65 |
| Craig_FSM_022 | FFPE/FSM | 0.21 | 35.59 | 21.44 | 171.23 | 135.08 | 4.81 | 6.30 | 18.35 | 19.05 |
| Craig_FSM_023 | FFPE/FSM | 0.19 | 36.84 | 29.38 | 192.58 | 152.76 | 5.23 | 5.20 | 12.00 | 12.80 |
| Craig_FSM_024 | FFPE/FSM | 0.29 | 41.73 | 18.39 | 172.75 | 82.61 | 4.14 | 4.49 | 13.77 | 16.19 |
| Craig_FSM_025 | FFPE/FSM | 0.22 | 48.57 | 27.63 | 221.36 | 153.88 | 4.56 | 5.57 | 13.00 | 14.02 |
| Craig_FSM_026 | FFPE/FSM | 0.23 | 34.64 | 17.27 | 176.07 | 97.36 | 5.05 | 5.64 | 14.10 | 17.58 |
| Craig_FSM_027 | FFPE/FSM | 0.20 | 36.66 | 22.76 | 164.50 | 128.46 | 4.49 | 5.64 | 16.27 | 17.97 |
| Craig_FSM_028 | FFPE/FSM | 0.22 | 39.18 | 19.88 | 160.16 | 119.70 | 4.28 | 6.02 | 15.75 | 17.39 |
| Craig_FSM_029 | FFPE/FSM | 0.20 | 34.01 | 20.70 | 162.17 | 142.79 | 4.77 | 6.90 | 15.18 | 16.64 |
| Craig_FSM_030 | FFPE/FSM | 0.20 | 43.97 | 28.28 | 180.75 | 153.59 | 4.32 | 5.43 | 16.32 | 17.80 |
| Craig_FSM_031 | FFPE/FSM | 0.21 | 41.16 | 30.81 | 220.08 | 201.46 | 5.35 | 6.60 | 15.25 | 16.73 |
| Craig_FSM_032 | FFPE/FSM | 0.16 | 44.44 | 36.67 | 201.90 | 206.26 | 4.54 | 3.62 | 15.62 | 17.33 |
| Craig_FSM_033 | FFPE/FSM | 0.17 | 40.00 | 31.74 | 168.09 | 146.33 | 4.20 | 4.61 | 13.17 | 15.97 |
| Craig_FSM_034 | FFPE/FSM | 0.15 | 48.80 | 41.00 | 223.44 | 214.32 | 4.58 | 3.23 | 15.91 | 17.32 |
| Craig_FSM_035 | FFPE/FSM | 0.27 | 41.26 | 18.00 | 224.55 | 113.34 | 5.44 | 6.30 | 13.21 | 15.45 |
| Craig_FSM_036 | FFPE/FSM | 0.18 | 39.72 | 27.13 | 190.51 | 125.98 | 4.80 | 4.61 | 14.63 | 16.29 |
| Craig_FSM_037 | FFPE/FSM | 0.16 | 46.37 | 31.70 | 229.14 | 148.46 | 4.94 | 4.68 | 15.13 | 16.43 |
| Craig_FSM_038 | FFPE/FSM | 0.20 | 49.86 | 25.23 | 250.70 | 102.96 | 5.03 | 4.08 | 12.40 | 17.99 |
| Craig_FSM_039 | FFPE/FSM | 0.17 | 41.61 | 28.75 | 151.10 | 123.63 | 3.63 | 4.10 | 13.07 | 15.73 |
| Craig_FSM_040 | FFPE/FSM | 0.21 | 45.39 | 29.65 | 227.55 | 159.53 | 5.01 | 5.38 | 16.55 | 16.83 |
| Craig_FSM_041 | FFPE/FSM | 0.17 | 46.25 | 30.28 | 218.29 | 160.40 | 4.72 | 5.30 | 13.29 | 17.24 |
| Craig_FSM_042 | FFPE/FSM | 0.17 | 51.73 | 30.85 | 223.21 | 169.56 | 4.32 | 5.50 | 14.37 | 17.32 |
| Craig_FSM_043 | FFPE/FSM | 0.16 | 41.04 | 30.46 | 175.09 | 165.41 | 4.27 | 5.43 | 15.60 | 17.32 |
| Craig_FSM_044 | FFPE/FSM | 0.20 | 46.60 | 31.05 | 215.07 | 162.46 | 4.01 | 5.26 | 13.75 | 15.75 |
| Craig_FSM_045 | FFPE/FSM | 0.24 | 50.44 | 31.30 | 151.64 | 132.10 | 3.01 | 4.22 | 12.39 | 13.11 |
| Craig_FSM_046 | FFPE/FSM | 0.27 | 53.91 | 31.01 | 159.90 | 144.08 | 2.97 | 4.65 | 14.39 | 13.37 |
| Craig_FSM_047 | FFPE/FSM | 0.26 | 60.17 | 36.09 | 164.05 | 143.59 | 2.73 | 3.98 | 13.75 | 13.18 |
| Craig_FSM_048 | FFPE/FSM | 0.19 | 47.82 | 36.44 | 149.55 | 101.05 | 3.13 | 4.42 | 12.94 | 13.54 |
| Craig_FSM_049 | FFPE/FSM | 0.22 | 38.76 | 25.18 | 129.40 | 118.12 | 3.34 | 4.70 | 13.40 | 13.95 |
| Craig_FSM_050 | FFPE/FSM | 0.22 | 33.93 | 24.40 | 124.97 | 130.18 | 3.68 | 5.33 | 14.67 | 13.66 |
| Craig_FSM_051 | FFPE/FSM | 0.21 | 43.84 | 32.39 | 132.09 | 132.78 | 3.01 | 4.10 | 14.49 | 14.10 |
| Craig_FSM_052 | FFPE/FSM | 0.23 | 36.06 | 22.49 | 104.76 | 90.36 | 2.90 | 4.02 | 14.87 | 13.79 |
| Craig_FSM_053 | FFPE/FSM | 0.20 | 47.34 | 38.58 | 138.59 | 177.27 | 2.93 | 4.60 | 14.12 | 14.90 |
| Craig_FSM_054 | FFPE/FSM | 0.25 | 25.30 | 22.60 | 90.72 | 83.87 | 2.74 | 3.71 | 16.06 | 17.33 |
| Craig_FSM_055 | FFPE/FSM | 0.23 | 28.00 | 28.66 | 112.54 | 167.89 | 4.02 | 5.86 | 14.14 | 15.48 |
| Craig_FSM_056 | FFPE/FSM | 0.26 | 26.84 | 26.20 | 77.34 | 118.44 | 2.88 | 4.52 | 15.24 | 16.48 |
| Craig_FSM_057 | FFPE/FSM | 0.24 | 33.54 | 20.74 | 102.95 | 93.65 | 3.17 | 4.52 | 15.63 | 17.73 |
| Craig_FSM_058 | FFPE/FSM | 0.20 | 45.05 | 36.67 | 142.86 | 163.39 | 3.17 | 4.46 | 10.90 | 12.54 |
| Craig_FSM_059 | FFPE/FSM | 0.24 | 22.35 | 19.24 | 164.84 | 106.38 | 7.38 | 5.08 | 17.73 | 11.89 |
| Craig_FSM_060 | FFPE/FSM | 0.20 | 28.36 | 25.44 | 200.81 | 118.64 | 7.08 | 4.66 | 15.40 | 14.99 |
| Craig_FSM_061 | FFPE/FSM | 0.20 | 32.66 | 29.27 | 211.65 | 148.93 | 6.48 | 5.09 | 18.10 | 17.99 |
| Craig_FSM_062 | FFPE/FSM | 0.17 | 38.18 | 26.63 | 145.11 | 139.59 | 3.80 | 5.24 | 14.75 | 15.20 |
| Craig_FSM_063 | FFPE/FSM | 0.16 | 39.50 | 21.95 | 162.56 | 167.60 | 4.12 | 5.26 | 14.59 | 14.40 |
| Craig_FSM_064 | FFPE/FSM | 0.16 | 43.28 | 36.73 | 170.73 | 192.71 | 3.94 | 5.26 | 14.64 | 14.76 |
| Craig_FSM_065 | FFPE/FSM | 0.18 | 35.04 | 33.09 | 140.07 | 164.02 | 4.00 | 4.96 | 17.13 | 15.99 |
| Craig_FSM_066 | FFPE/FSM | 0.16 | 44.55 | 36.46 | 179.01 | 185.32 | 4.02 | 5.68 | 14.70 | 14.89 |
| Craig_FSM_067 | FFPE/FSM | 0.20 | 36.50 | 23.45 | 152.24 | 109.08 | 4.17 | 4.65 | 14.97 | 17.18 |
| Craig_FSM_068 | FFPE/FSM | 0.24 | 34.57 | 26.00 | 128.30 | 117.36 | 3.71 | 4.51 | 16.88 | 17.12 |
| Craig_FSM_069 | FFPE/FSM | 0.15 | 44.49 | 33.77 | 175.56 | 169.11 | 3.95 | 5.01 | 15.54 | 15.99 |
| Craig_FSM_070 | FFPE/FSM | 0.19 | 40.23 | 26.46 | 179.74 | 127.13 | 4.47 | 4.99 | 15.85 | 17.28 |
| Craig_FSM_071 | FFPE/FSM | 0.17 | 40.85 | 29.75 | 164.88 | 149.88 | 4.04 | 5.04 | 16.03 | 17.37 |
| Craig_FSM_072 | FFPE/FSM | 0.18 | 45.00 | 34.97 | 173.48 | 179.13 | 3.85 | 5.12 | 16.84 | 16.44 |
| Craig_FSM_073 | FFPE/FSM | 0.22 | 38.85 | 22.90 | 150.45 | 108.42 | 3.87 | 4.74 | 14.60 | 15.89 |
| Craig_FSM_074 | FFPE/FSM | 0.19 | 33.72 | 20.31 | 137.98 | 157.62 | 4.09 | 5.20 | 16.20 | 16.58 |
| Craig_FSM_075 | FFPE/FSM | 0.18 | 33.96 | 29.03 | 136.18 | 166.10 | 4.01 | 5.72 | 16.41 | 16.3 |
| Craig_FSM_076 | FFPE/FSM | 0.18 | 34.27 | 31.59 | 140.95 | 150.32 | 4.11 | 4.76 | 16.84 | 15.68 |
| Craig_FSM_077 | FFPE/FSM | 0.21 | 33.93 | 30.94 | 122.74 | 151.66 | 3.62 | 4.90 | 15.40 | 17.64 |
| Craig_FSM_078 | FFPE/FSM | 0.22 | 24.31 | 22.89 | 130.84 | 124.32 | 5.28 | 5.43 | 15.61 | 16.64 |
| Craig_FSM_079 | FFPE/FSM | 0.21 | 27.48 | 30.34 | 141.45 | 198.59 | 5.15 | 6.56 | 14.96 | 16.75 |
| Craig_FSM_080 | FFPE/FSM | 0.18 | 30.43 | 34.55 | 143.63 | 192.78 | 4.72 | 3.58 | 15.68 | 15.34 |
| Craig_FSM_081 | FFPE/STANDARD | 0.78 | 2.66 | 6.16 | 8.54 | 29.55 | 3.21 | 4.80 | na | 22.97 |
| Craig_FSM_082 | FFPE/STANDARD | 0.31 | 20.11 | 11.4 | 61.54 | 51.33 | 3.06 | 4.40 | 20.16 | 22.17 |
| Craig_FSM_083 | FFPE/STANDARD | 0.36 | 16.02 | 13.48 | 39.64 | 51.92 | 2.47 | 3.85 | 22.48 | 24.17 |
| Craig_FSM_084 | FFPE/STANDARD | 0.32 | 11.72 | 13.52 | 36.47 | 73.25 | 3.31 | 5.42 | 21.62 | 19.63 |
| Craig_FSM_085 | FFPE/STANDARD | 0.48 | 8.77 | 9.96 | 28.98 | 80.07 | 3.30 | 8.04 | 57.35 | 55.47 |
| Craig_FSM_086 | FFPE/STANDARD | 0.28 | 13.98 | 22.72 | 47.43 | 131.05 | 3.39 | 5.77 | 19.15 | 16.14 |
| Craig_FSM_087 | FFPE/STANDARD | 0.34 | 11.20 | 27.64 | 34.76 | 162.79 | 3.10 | 5.89 | 22.43 | 20.68 |
| Craig_FSM_088 | FFPE/STANDARD | 0.40 | 14.39 | 8.07 | 80.94 | 90.39 | 4.18 | 11.22 | 23.52 | 22.11 |
| Craig_FSM_089 | FFPE/STANDARD | 0.46 | 6.96 | 13.28 | 28.86 | 119.81 | 4.15 | 9.04 | 22.93 | 21.75 |
| Craig_FSM_090 | FFPE/STANDARD | 0.65 | 5.50 | 6.44 | 19.80 | 40.56 | 3.60 | 6.30 | 37.81 | 32.39 |
| Craig_FSM_091 | FFPE/STANDARD | 0.37 | 9.61 | 11.37 | 30.57 | 58.98 | 3.18 | 5.19 | 21.89 | 20.32 |
| Craig_FSM_092 | FFPE/STANDARD | 0.27 | 20.69 | 18.14 | 81.81 | 84.33 | 3.95 | 4.65 | 19.89 | 20.27 |
| Craig_FSM_093 | FFPE/STANDARD | 0.35 | 20.68 | 9.77 | 60.38 | 37.37 | 2.92 | 3.82 | 20.33 | 23.78 |
| Craig_FSM_094 | FFPE/STANDARD | 0.30 | 14.28 | 27.48 | 41.18 | 134.96 | 2.88 | 4.91 | 19.03 | 18.89 |
| Craig_FSM_095 | FFPE/STANDARD | 0.63 | 2.89 | 4.48 | 23.22 | 18.28 | 2.94 | 4.08 | 21.08 | 24.73 |
| Craig_FSM_096 | FFPE/STANDARD | 0.35 | 11.84 | 20.21 | 41.43 | 123.76 | 3.50 | 6.22 | 21.75 | 19.11 |
| Craig_FSM_097 | FFPE/STANDARD | 0.24 | 55.27 | 19.40 | 208.48 | 111.46 | 3.77 | 5.75 | 15.17 | 17.51 |
| Craig_FSM_098 | FFPE/STANDARD | 0.27 | 54.84 | 30.48 | 413.50 | 219.16 | 2.54 | 7.19 | 15.62 | 19.26 |
| Craig_FSM_099 | FFPE/STANDARD | 0.22 | 52.31 | 20.99 | 177.81 | 94.04 | 3.40 | 4.48 | 16.60 | 18.79 |
| Craig_FSM_100 | FFPE/STANDARD | 0.20 | 56.18 | 22.19 | 224.52 | 127.30 | 4.07 | 5.74 | 15.74 | 18.09 |
| Craig_FSM_101 | FFPE/STANDARD | 0.32 | 10.80 | 19.06 | 22.47 | 113.92 | 3.01 | 5.98 | 19.95 | 18.97 |
| Craig_FSM_102 | FFPE/STANDARD | 0.37 | 9.59 | 22.47 | 37.40 | 161.26 | 3.90 | 6.73 | 14.36 | 13.53 |
| Craig_FSM_103 | FFPE/STANDARD | 0.43 | 40.94 | 14.83 | 135.33 | 66.67 | 3.31 | 6.50 | 56.13 | 54.50 |
| Craig_FSM_104 | FFPE/STANDARD | 0.46 | 19.14 | 22.20 | 275.65 | 120.82 | 14.40 | 3.43 | 12.34 | 12.62 |
| Craig_FSM_105 | FFPE/STANDARD | 0.37 | 17.90 | 25.51 | 768.87 | 196.79 | 42.95 | 7.71 | 16.81 | 20.42 |
| Craig_FSM_106 | FFPE/STANDARD | 0.31 | 16.48 | 25.14 | 62.36 | 191.09 | 3.78 | 7.60 | 14.52 | 9.18 |
| Craig_FSM_107 | FFPE/STANDARD | 0.29 | 48.05 | 37.29 | 478.60 | 227.57 | 9.96 | 6.30 | 15.59 | 16.71 |
| Craig_FSM_108 | FFPE/STANDARD | 0.26 | 16.68 | 29.05 | 106.93 | 155.48 | 6.41 | 5.35 | 13.62 | 13.57 |
| Craig_FSM_109 | FFPE/STANDARD | 0.24 | 26.75 | 34.08 | 108.1 | 225.91 | 4.04 | 6.63 | 14.13 | 15.36 |
| Craig_FSM_110 | FFPE/STANDARD | 0.21 | 16.20 | 43.94 | 117.75 | 275.64 | 7.27 | 6.27 | 13.94 | 12.72 |
| Craig_FSM_111 | FFPE/STANDARD | 0.32 | 21.76 | 32.08 | 75.63 | 210.01 | 3.48 | 6.55 | 15.03 | 15.83 |
| Craig_FSM_112 | FFPE/STANDARD | 0.40 | 19.85 | 21.15 | 85.77 | 125.86 | 4.32 | 5.95 | 15.44 | 16.66 |
| Craig_FSM_113 | FFPE/STANDARD | 0.33 | 16.25 | 19.18 | 78.29 | 152.43 | 4.82 | 7.95 | 15.44 | 114.03 |
| Craig_FSM_114 | FFPE/STANDARD | 0.34 | 10.64 | 24.69 | 74.73 | 148.46 | 3.81 | 6.01 | 18.28 | 17.33 |
| Craig_FSM_115 | FFPE/STANDARD | 0.32 | 20.09 | 28.71 | 76.50 | 203.10 | 3.81 | 7.97 | 16.88 | 15.25 |
| Craig_FSM_116 | FFPE/STANDARD | 0.31 | 41.20 | 32.05 | 441.25 | 214.59 | 10.71 | 6.70 | 17.23 | 17.52 |
| Craig_FSM_117 | FFPE/STANDARD | 0.36 | 16.67 | 21.12 | 63.12 | 187.62 | 3.79 | 8.88 | 16.11 | 12.51 |
| Craig_FSM_118 | FFPE/STANDARD | 0.46 | 21.74 | 28.11 | 89.95 | 210.44 | 4.14 | 7.49 | 13.39 | 15.90 |
| Craig_FSM_119 | FFPE/STANDARD | 0.31 | 48.06 | 10.83 | 195.13 | 28.97 | 4.06 | 2.67 | 13.93 | 21.34 |
| Craig_FSM_120 | FFPE/STANDARD | 0.26 | 18.83 | 33.49 | 77.73 | 198.54 | 4.13 | 5.93 | 19.03 | 21.31 |
| Craig_FSM_121 | FFPE/STANDARD | 0.47 | 12.58 | 11.34 | 45.73 | 46.57 | 3.63 | 4.10 | 21.45 | 24.24 |
| Craig_FSM_122 | FFPE/STANDARD | 0.57 | 39.58 | 6.63 | 419.86 | 18.44 | 7.05 | 2.78 | 14.59 | 23.72 |
| Craig_FSM_123 | CELLS/FSM | 0.20 | 31.31 | 29.73 | 122.14 | 168.24 | 3.60 | 5.66 | 17.61 | 19.12 |
| Craig_FSM_124 | CELLS/FSM | 0.18 | 39.37 | 40.06 | 138.52 | 208.21 | 3.52 | 5.20 | 16.06 | 16.47 |
| Craig_FSM_125 | CELLS/FSM | 0.18 | 42.90 | 46.69 | 176.89 | 285.94 | 4.12 | 6.12 | 14.65 | 12.86 |
| Craig_FSM_126 | CELLS/FSM | 0.18 | 40.03 | 39.84 | 173.97 | 250.32 | 4.35 | 6.28 | 15.57 | 15.47 |
| Craig_FSM_127 | CELLS/FSM | 0.18 | 42.03 | 46.22 | 129.26 | 214.52 | 3.08 | 4.64 | 15.20 | 14.00 |
| Craig_FSM_128 | CELLS/FSM | 0.22 | 36.83 | 26.27 | 125.09 | 122.18 | 3.40 | 4.65 | 15.03 | 15.18 |
| Craig_FSM_129 | CELLS/FSM | 0.15 | 44.51 | 42.71 | 184.17 | 249.88 | 4.14 | 5.85 | 14.47 | 13.48 |
| Craig_FSM_130 | CELLS/FSM | 0.21 | 48.89 | 28.24 | 352.25 | 153.44 | 7.21 | 5.43 | 15.91 | 14.48 |
| Craig_FSM_131 | CELLS/FSM | 0.22 | 35.76 | 27.16 | 127.49 | 123.91 | 3.57 | 4.56 | 14.82 | 14.57 |
| Craig_FSM_132 | CELLS/FSM | 0.19 | 31.18 | 27.14 | 91.46 | 127.64 | 2.93 | 4.70 | 16.28 | 16.15 |
| Craig_FSM_133 | CELLS/FSM | 0.18 | 42.14 | 45.66 | 131.94 | 219.34 | 3.13 | 4.80 | 15.95 | 14.48 |
| Craig_FSM_134 | CELLS/FSM | 0.18 | 29.67 | 25.12 | 127.78 | 136.13 | 4.31 | 5.42 | 14.01 | 14.77 |
| Craig_FSM_135 | CELLS/FSM | 0.18 | 53.16 | 50.55 | 160.25 | 229.82 | 3.01 | 4.55 | 14.83 | 15.65 |
| Craig_FSM_136 | CELLS/FSM | 0.28 | 30.91 | 28.26 | 136.54 | 122.29 | 4.42 | 4.33 | 15.23 | 14.84 |
| Craig_FSM_137 | CELLS/FSM | 0.18 | 32.80 | 27.76 | 176.19 | 109.87 | 3.34 | 3.96 | 15.88 | 21.85 |
| Craig_FSM_138 | CELLS/FSM | 0.20 | 38.61 | 27.58 | 126.95 | 116.55 | 3.13 | 4.23 | 14.19 | 15.68 |
| Craig_FSM_139 | CELLS/FSM | 0.22 | 57.95 | 39.14 | 180.25 | 194.82 | 3.11 | 4.98 | 14.51 | 14.23 |
| Craig_FSM_140 | CELLS/FSM | 0.24 | 16.69 | 17.91 | 127.59 | 119.92 | 7.64 | 6.19 | 18.34 | 18.52 |
| Craig_FSM_141 | CELLS/FSM | 0.15 | 31.69 | 43.90 | 255.28 | 259.80 | 8.05 | 5.92 | 14.91 | 15.29 |
| Craig_FSM_142 | CELLS/FSM | 0.23 | 16.42 | 21.65 | 115.79 | 121.36 | 7.05 | 5.61 | 16.20 | 13.75 |
| Craig_FSM_143 | CELLS/FSM | 0.22 | 17.84 | 21.30 | 144.17 | 172.52 | 8.08 | 8.10 | 14.36 | 14.66 |
| Craig_FSM_144 | CELLS/FSM | 0.22 | 27.31 | 20.06 | 80.26 | 95.06 | 2.94 | 4.74 | 22.86 | 22.31 |
| Craig_FSM_145 | CELLS/FSM | 0.18 | 36.31 | 41.74 | 144.93 | 206.09 | 3.99 | 5.95 | 15.81 | 16.45 |
| Craig_FSM_146 | CELLS/FSM | 0.17 | 32.41 | 40.69 | 136.34 | 228.72 | 4.21 | 5.62 | 16.28 | 16.10 |
| Craig_FSM_147 | CELLS/FSM | 0.18 | 28.42 | 27.70 | 137.76 | 150.95 | 4.85 | 5.45 | 15.90 | 16.28 |
| Craig_FSM_148 | CELLS/FSM | 0.17 | 33.54 | 38.56 | 139.79 | 235.22 | 4.17 | 6.10 | 14.97 | 14.50 |
| Craig_FSM_149 | CELLS/FSM | 0.15 | 31.82 | 36.39 | 154.85 | 248.69 | 4.87 | 6.83 | 15.28 | 16.49 |
| Craig_FSM_150 | CELLS/FSM | 0.16 | 33.34 | 38.46 | 140.80 | 216.67 | 4.22 | 5.63 | 18.27 | 18.42 |
| Craig_FSM_151 | CELLS/FSM | 0.22 | 43.44 | 43.54 | 179.25 | 312.19 | 4.13 | 2.17 | 13.70 | 13.00 |
| Craig_FSM_152 | CELLS/FSM | 0.18 | 43.30 | 41.01 | 183.08 | 299.20 | 4.23 | 2.17 | 12.90 | 12.44 |
| Craig_FSM_153 | FROZEN/FSM | 0.15 | 59.64 | 37.95 | 478.67 | 216.58 | 5.03 | 5.71 | 15.80 | 18.75 |
| Craig_FSM_154 | FROZEN/FSM | 0.15 | 66.70 | 40.00 | 473.78 | 221.76 | 7.10 | 5.54 | 17.52 | 19.77 |
| Craig_FSM_155 | FROZEN/FSM | 0.13 | 87.30 | 57.19 | 485.24 | 292.00 | 5.56 | 5.12 | 15.36 | 17.91 |
| Craig_FSM_156 | FROZEN/FSM | 0.13 | 72.73 | 50.87 | 440.45 | 263.40 | 6.06 | 5.18 | 16.14 | 17.75 |
| Craig_FSM_157 | FROZEN/FSM | 0.12 | 68.22 | 50.73 | 411.76 | 285.23 | 6.04 | 5.62 | 15.11 | 17.69 |
| Craig_FSM_158 | FROZEN/FSM | 0.14 | 68.40 | 43.06 | 395.43 | 209.96 | 5.78 | 4.88 | 16.57 | 18.42 |
| Craig_FSM_159 | FROZEN/FSM | 0.14 | 60.54 | 42.68 | 353.33 | 228.84 | 5.08 | 5.18 | 15.48 | 16.49 |
| Craig_FSM_160 | FROZEN/FSM | 0.14 | 62.19 | 44.88 | 430.03 | 250.88 | 6.91 | 5.50 | 17.56 | 21.03 |
| Craig_FSM_161 | CELLS/FSM | 0.15 | 51.61 | 43.44 | 254.25 | 200.40 | 4.93 | 4.61 | 30.93 | 13.38 |
| Craig_FSM_162 | CELLS/FSM | 0.17 | 57.57 | 40.55 | 297.11 | 209.84 | 5.16 | 5.18 | 16.71 | 16.97 |
| Craig_FSM_163 | CELLS/FSM | 0.16 | 56.85 | 47.08 | 311.82 | 236.94 | 5.48 | 5.03 | 15.29 | 15.93 |
| Craig_FSM_164 | CELLS/FSM | 0.21 | 40.07 | 24.26 | 150.20 | 95.34 | 3.75 | 3.93 | 13.59 | 16.07 |
| Craig_FSM_165 | CELLS/FSM | 0.16 | 57.14 | 20.86 | 258.37 | 175.52 | 4.52 | 4.40 | 14.91 | 16.46 |
| Craig_FSM_166 | CELLS/FSM | 0.16 | 54.74 | 24.84 | 252.47 | 169.01 | 4.61 | 4.56 | 12.09 | 12.58 |
| Craig_FSM_167 | CELLS/FSM | 0.26 | 23.55 | 16.08 | 75.63 | 56.75 | 3.21 | 3.53 | 13.09 | 16.26 |
| Craig_FSM_168 | CELLS/FSM | 0.17 | 50.45 | 47.23 | 289.29 | 312.77 | 5.73 | 6.62 | 14.15 | 14.08 |
| Craig_FSM_169 | CELLS/FSM | 0.15 | 49.10 | 41.69 | 226.72 | 230.64 | 4.62 | 5.53 | 13.98 | 15.51 |
| Craig_FSM_170 | CELLS/FSM | 0.16 | 38.86 | 36.07 | 230.83 | 206.72 | 5.84 | 5.73 | 12.31 | 14.68 |
| Craig_FSM_171 | CELLS/FSM | 0.17 | 43.26 | 34.78 | 221.08 | 219.95 | 5.11 | 6.32 | 11.35 | 13.14 |
| Craig_FSM_172 | CELLS/STANDARD | 0.19 | 46.18 | 30.81 | 212.89 | 192.95 | 4.61 | 6.26 | 13.28 | 13.04 |
| Craig_FSM_173 | CELLS/STANDARD | 0.21 | 51.04 | 68.12 | 320.95 | 853.62 | 6.29 | 12.53 | 14.88 | 10.67 |
| Craig_FSM_174 | CELLS/STANDARD | 0.21 | 48.89 | 28.24 | 352.25 | 153.44 | 7.21 | 5.43 | 15.01 | 14.48 |
| Craig_FSM_175 | CELLS/STANDARD | 0.22 | 45.43 | 53.38 | 284.32 | 349.15 | 6.25 | 6.54 | 15.03 | 15.77 |
| Craig_FSM_176 | CELLS/STANDARD | 0.24 | 36.26 | 23.25 | 360.65 | 154.35 | 4.68 | 6.64 | 11.73 | 11.11 |
| Craig_FSM_177 | CELLS/STANDARD | 0.24 | 41.77 | 42.97 | 330.38 | 417.35 | 7.91 | 9.71 | 12.83 | 12.48 |
| Craig_FSM_178 | CELLS/STANDARD | 0.24 | 48.82 | 37.17 | 282.22 | 173.36 | 5.78 | 4.66 | 14.06 | 13.72 |
| Craig_FSM_179 | CELLS/STANDARD | 0.26 | 24.08 | 45.38 | 85.34 | 290.96 | 3.54 | 6.41 | 16.15 | 13.98 |
| Craig_FSM_180 | CELLS/STANDARD | 0.26 | 26.33 | 38.72 | 238.29 | 327.87 | 8.07 | 8.73 | 19.20 | 18.22 |
| Craig_FSM_181 | CELLS/STANDARD | 0.26 | 39.64 | 39.29 | 300.21 | 310.63 | 7.57 | 7.91 | 14.48 | 15.22 |
| Craig_FSM_182 | CELLS/STANDARD | 0.28 | 34.79 | 33.47 | 261.42 | 291.51 | 7.51 | 8.77 | 14.54 | 12.42 |
| Craig_FSM_183 | CELLS/STANDARD | 0.28 | 27.25 | 20.69 | 162.08 | 212.26 | 3.98 | 2.15 | 28.47 | 24.32 |
| Craig_FSM_184 | CELLS/STANDARD | 0.29 | 43.25 | 46.64 | 284.08 | 344.73 | 6.91 | 2.39 | 13.31 | 13.22 |
| Craig_FSM_185 | CELLS/STANDARD | 0.29 | 30.21 | 17.77 | 154.30 | 86.06 | 3.11 | 4.89 | 13.06 | 17.11 |
| Craig_FSM_186 | CELLS/STANDARD | 0.31 | 32.74 | 33.67 | 121.08 | 195.12 | 3.70 | 5.80 | 25.75 | 21.56 |
| Craig_FSM_187 | CELLS/STANDARD | 0.31 | 9.83 | 24.86 | 77.57 | 132.27 | 7.80 | 5.32 | 17.97 | 16.56 |
| Craig_FSM_188 | CELLS/STANDARD | 0.32 | 35.63 | 28.27 | 121.20 | 167.17 | 1.40 | 5.91 | 16.95 | 18.89 |
| Craig_FSM_189 | CELLS/STANDARD | 0.32 | 25.26 | 23.19 | 116.53 | 182.08 | 4.61 | 7.85 | 20.00 | 21.31 |
| Craig_FSM_190 | CELLS/STANDARD | 0.32 | 43.14 | 26.13 | 245.53 | 127.05 | 5.00 | 4.86 | 26.29 | 16.93 |
| Craig_FSM_191 | CELLS/STANDARD | 0.33 | 8.48 | 33.20 | 79.18 | 198.35 | 9.34 | 5.97 | 17.25 | 14.83 |
| Craig_FSM_192 | CELLS/STANDARD | 0.34 | 38.87 | 33.92 | 215.73 | 189.52 | 6.32 | 5.59 | 31.65 | 20.23 |
| Craig_FSM_193 | CELLS/STANDARD | 0.35 | 30.84 | 23.30 | 155.66 | 192.73 | 5.05 | 5.79 | 29.85 | 13.04 |
| Craig_FSM_194 | CELLS/STANDARD | 0.39 | 38.83 | 18.36 | 174.74 | 113.85 | 4.50 | 6.20 | 84.64 | 72.63 |
| Craig_FSM_195 | CELLS/STANDARD | 0.40 | 24.84 | 15.20 | 86.84 | 102.70 | 3.52 | 6.76 | 16.65 | 16.93 |
| Craig_FSM_196 | CELLS/STANDARD | 0.43 | 27.25 | 19.33 | 119.18 | 118.20 | 4.37 | 6.11 | 35.92 | 33.60 |
| Craig_FSM_197 | CELLS/STANDARD | 0.58 | 21.39 | 5.28 | 58.79 | 18.52 | 2.75 | 2.51 | 18.45 | 21.12 |
| Craig_FSM_198 | CELLS/STANDARD | 0.63 | 3.24 | 11.53 | 22.81 | 56.95 | 2.04 | 4.93 | 20.16 | 13.65 |
| Craig_FSM_199 | CELLS/STANDARD | 0.68 | 20.99 | 1.41 | 58.47 | 6.55 | 2.79 | 4.65 | 18.10 | na |
| Craig_FSM_200 | CELLS/STANDARD | 0.81 | 24.07 | 1.78 | 80.22 | 7.32 | 3.33 | 4.10 | 14.98 | na |

What is claimed:

1. A method of generating nucleic acid fragments having a customized fragment size distribution, comprising:
a) obtaining a master pool of nucleic acid molecules to be fragmented;
b) fragmenting at least two independent aliquots of the master pool of nucleic acid molecules in separate reactions, wherein the fragmentation conditions of each separate reaction are identical except for a single variable;
c) determining the nucleic acid molecule fragment size distribution from each aliquot;
d) plotting each nucleic acid molecule fragment size distribution result on a graph as a function of a value of the single variable for each aliquot;
e) fitting a curve to the plotted nucleic acid molecule fragment size distribution results;
f) identifying the value of the single variable necessary to obtain the desired nucleic acid molecule fragment size distribution on the curve; and
g) fragmenting the master pool of nucleic acid molecules or an aliquot thereof, wherein the fragmentation conditions are performed using the identified value of the single variable necessary to obtain the desired nucleic acid molecule fragment size distribution, to thereby generate nucleic acid fragments having a customized fragment size distribution.

2. The method of claim 1, wherein step b) further comprises treating the nucleic acid molecules or fragments thereof with at least one additional nucleic acid modifying reaction to modify or simulate the modification of the nucleic acid molecules or fragments thereof.

3. The method of claim 2, wherein the at least one additional nucleic acid modifying reaction is a nucleic acid labeling reaction.

4. The method of claim 2 or 3, wherein the at least one additional nucleic acid modifying reaction or simulated reaction thereof is performed before, simultaneously with, or after the fragmentation reaction.

5. The method of any one of claims 2-4, wherein step g) further comprises treating the nucleic acid fragments with the at least one additional nucleic acid modifying reaction of step b).

6. The method of claim 5, wherein the at least one additional nucleic acid modifying reaction is a nucleic acid labeling reaction.

7. The method of claim 5, wherein the at least one additional nucleic acid modifying reaction is performed before, simultaneously with, or after the fragmentation reaction.

8. The method of claim 1, wherein the nucleic acid fragments having a customized fragment size distribution are used in a nucleic acid hybridization, sequencing, or amplification assay and step b) further comprises treating the nucleic acid molecules or fragments thereof with every nucleic acid processing step required for the assay prior to hybridization, sequencing, or amplification, or modeling each step thereof.

9. The method of claim 8, wherein the nucleic acid processing or modeled processing steps are performed before, simultaneously with, or after the fragmentation reaction.

10. The method of claim 8 or 9, wherein step g) further comprises treating the nucleic acid fragments thereof with every nucleic acid processing step required for the assay prior to hybridization, sequencing, or amplification.

11. The method of any one of claims 8-10, wherein the nucleic acid processing steps are performed before, simultaneously with, or after the fragmentation reaction.

12. The method of claim 1, wherein the nucleic acid molecules are obtained from a sample selected from the group consisting of formalin-fixed paraffin-embedded (FFPE), paraffin, frozen, and fresh samples.

13. The method of claim 12, wherein the sample contains a tissue specimen and the tissue specimen was present in the sample for more than one year after isolation from a host organism.

14. The method of claim 1, wherein the nucleic acid molecules to be fragmented are selected from the group consisting of genomic DNA, cDNA, double-stranded DNA, single-stranded DNA, double-stranded RNA, single-stranded RNA, and messenger RNAs.

15. The method of claim 1, 2, or 7, wherein the nucleic acid molecules to be fragmented are fragmented by heat fragmentation, enzymatic digestion, shearing, mechanical crushing, chemical treatment, nebulizing, or sonication.

16. The method of claim 1, 2, or 7, wherein the single variable is selected from the group consisting of time, temperature, pressure, shear force, reagent amount, reagent concentration, reagent activity, acoustic wavelength, and acoustic frequency.

17. The method of claim 1, 2, or 7, wherein the at least two aliquots of step b) are performed simultaneously or sequentially.

18. The method of claim 1, 2, or 7, wherein step b) is performed with at least 3 or at least 4 aliquots.

19. The method of claim 1, 2, or 7, wherein the fragment size distribution is measured as the mode, mean, or median of fragment lengths.

20. The method of claim 1, 2, or 7, wherein the curve is fit using a linear model, an exponential decay model, or an inverse power law.

21. The method of claim 20, wherein the inverse power law is given by the mathematical formula,
$$f ( t ) = {θ}_{1} + \frac{{θ}_{2}}{{( t + {θ}_{3} )}^{{θ}_{4}}} ,$$
where f(t) is the mode DNA fragment size, t is the single variable for each aliquot representing time of heat fragmentation, and θ₁, θ₂, θ₃, and θ₄ are constant parameters unique for each aliquot.

22. The method of claim 21, wherein constant parameters, θ₁, θ₂, θ₃, and θ₄, are determined using iterative least squares non-linear regression.

23. A method of generating nucleic acid fragments having customized and essentially identical fragment size distributions from each of at least two independent master pools of nucleic acid molecules to be fragmented comprising performing the method of claim 1 using at least two master pools of nucleic acid molecules.
